(19) United States
(12) Patent Application Publication
(10) Pub. No.: US 2003/0104604 A1
(43) Pub. Date: Jun. 5, 2003

(54) Genetically engineered bacterial strains for the display of foreign peptides on filamentous phage

(76) Inventors: Weiping Yang, San Diego, CA (US); Ronald Somerville, West Lafayette, IN (US)

Correspondence Address: DAVID R PRESTON & ASSOCIATES, 12625 HIGH BLUFF DRIVE, SUITE 205, SAN DIEGO, CA, 92130, US

(21) Appl. No.: 10/211,296

(22) Filed: Aug. 2, 2002

Related U.S. Application Data
(60) Provisional application No. 60/310,171, filed on Aug. 3, 2001.

(51) Int. Cl. C12Q 1/70; C12N 7/00; C12N 1/21

(52) U.S. Cl. 435/235.1; 435/5; 435/252.3

(57) ABSTRACT

The present invention provides compositions and methods for increasing the efficiency of phage display. A first aspect of the present invention is a bacterial strain that stably comprises at least one gene that encodes a protein having a packaging function for a filamentous phage. A bacterial strain of the present invention can be transformed with a phage display expression construct that comprises a coat protein fusion protein or coat protein chimeric protein and used to generate packaged recombinant phage particles in the absence of a helper phage. In some preferred aspects of the invention, the bacterial strain contains ten of the eleven genes of an Ff filamentous phage that encode packaging functions, where a filamentous phage gene not contained by the bacterial strain is a coat protein gene that is provided on a phage display expression construct for the generation of fusion coat proteins or chimeric coat proteins. The present invention also includes methods of using a bacterial strain of the present invention to generate recombinant filamentous phage without the use of a helper phage. Preferred methods of the present invention include those that select for heavy chain and light chain antibody molecules with affinity for substances of interest. The invention also includes peptides and proteins identified using the methods of the present invention. Preferred peptides and proteins of the present invention include immunoglobulin heavy and light chains, and fragments thereof. Peptides or proteins selected for and identified using the methods of the present invention can be used for a variety of purposes, including research applications and therapeutic or diagnostic applications.

### CROSS-REFERENCE TO RELATED APPLICATIONS

This application claims benefit of priority to U.S. Provisional Application No. 60/310,171 filed Aug. 3, 2001, and naming Weiping Yang and Ronald Somerville as inventors, incorporated herein by reference in its entirety.

Sequence Listing

A sequence listing accompanies this application following the Abstract and is also provided electronically on a disk.

### BACKGROUND OF THE INVENTION

The field of the invention relates to the selection and identification of peptides and proteins with specific functions, particularly the selection and identification of peptides and proteins with specific functions from complex libraries, and in particular to the methodology known as phage display.

The filamentous phage are a group of bacteriophage characterized by having a single-stranded DNA genome that is encased by a long protein capsid cylinder. Filamentous phage infect gram-negative bacteria, among them, Pseudomonas aeruginosa, Vibrio parahaemolyticus, and the well-characterized and genetically manipulable E. coli. Bacteria must display an appendage (the sex pilus) encoded by the F plasmid, to be infected by filamentous phage. Infected bacteria are not lysed during the life cycle and replication of the phage, but rather experience a reduced rate of growth.

The best-known filamentous phage are those known as “Ff phage” that infect E. coli, including M13, fl, and fd. The genomes of these three phage are greater than 98% homologous, each of their genomes consisting of eleven genes (I-XI) that encode replication, assembly, and structural coat proteins that differ very little among themselves.

Because the filamentous phage can accommodate DNA sequences up to three times their natural size (the natural length of M13 is 6.4 kb), they have been used as cloning vehicles, especially in applications where it is desirable to isolate single-stranded DNA (for example, DNA sequencing, labeling, and mutagenesis). Thus, Messing and colleagues designed the “mp” series of M13 vectors for the isolation of single-stranded DNA, and subsequently constructed “phagemid” vectors that contained both plasmid and viral origins of replication (Viera and Messing, “Production of Single-Stranded Plasmid DNA” in Methods in Enzymology vol. 153, ed. Wu and Grossman, Academic Pres, New York, pp 3-11 (1987)). The phagemids incorporated advantages of both plasmids and phage: the double-standed plasmid form of the construct could be isolated in large quantities and manipulated with restriction enzymes, ligases, etc., whereas the single-stranded phage form of the construct could be readily isolated for sequencing, mutagenesis, or radiolabeling in reactions catalyzed by polymerases.

To generate single-stranded phage from phagemid vectors however, requires the use of a helper phage to supply replication, assembly, and extrusion functions (here referred to collectively as “packaging functions”) that are not carried on the phagemid. Thus “superinfection” with the helper phage promotes the synthesis of single-stranded virus from the phagemid template, which would otherwise be maintained within the cell in double-stranded form.

Superinfection of cells harboring phagemids with helper phage also causes a variable amount of the helper phage itself to become packaged as a filamentous phage, thus competing with phagemids in the assembly process. While this may not present a significant problem for many applications where single-stranded DNA is used (for example, in sequencing reactions where phagemid-specific primers can be used), it can cause significant interference in phage display experiments.

Phage display is an important method for the selection and identification of peptides and proteins with desirable properties. This method takes advantage of the fact that in filamentous phages, a particular nucleic acid molecule is physically linked, by virtue of the packaging process, to the peptides or proteins it encodes.

The Ff group of filamentous phage have been used in the development of vectors that allow fusions of peptides or proteins of interest with viral coat proteins. The coat proteins are incorporated into the phage particle along with peptide or protein sequences that can be retrieved by using, for example, panning procedures or assays. The selected phage particles, which optimally include the DNA that encodes the selected peptide or protein sequences, can be amplified, in the E. coli host and optionally subjected to additional rounds of selection. The selected particles can be isolated and the DNA sequence that encodes the peptide or protein of interest can be determined to identify peptides and proteins having desirable properties.

The efficiency of current phage display methodologies is less than optimal, however. The failure to capture phage particles that include DNA sequences that encode desirable peptide and protein variants can be attributed to two main deficiencies in the methods as currently practiced. Firstly, fusion or chimeric coat proteins encoded by a phagemid may never become incorporated into phage particles. Instead, packaged DNA may have only wild-type coat proteins associated with it. This is because, in addition to the gpIII fusion or chimeric protein encoded on a phage display expression vector, many phage display schemes also supply an additional wild-type cpIII protein in trans to promote infectivity of the resulting phage. The wild-type cpIII protein competes with the cpIII fusion or chimeric protein for binding to the assembling phage particle. Secondly, phage that contain desirable peptide or protein domains in the context of the phage particle may be displayed on phage and selected, but the captured phage particles may not contain the DNA that encodes the desirable sequences. Instead, a given particle might contain helper phage DNA that has preferentially packaged.

While attempts have been made to overcome these deficiencies, all of the current methodologies that use phagemid constructs employ helper phage to supply particular the required packaging functions in trans. The use helper phage has several drawbacks. Helper phage compete with phagemids for packaging into particles, they introduce added steps to the preparation of display phage, and they require considerable time and to maintain, prepare, and titer.

The present invention provides compositions and methods for improving the efficiency of methods for isolating and identifying peptide and protein sequences through phage display. The present invention provides other benefits as well.

### BRIEF SUMMARY OF THE INVENTION

The present invention recognizes the need to increase the efficiency of phage display methods that allow the isolation and identification of sequences that encode peptides and proteins with desirable properties. The present invention provides genetically engineered bacterial strains that stably comprise at least one gene that encodes a packaging function of a filamentous phage. The bacterial strain can be transformed with a phage display expression construct and can produce and package single-stranded DNA without the use of a helper phage. The present invention also provides methods for using a genetically engineered bacterial strain of the present invention to select and identify peptides and proteins having desirable properties. In preferred embodiments the methods of the present invention can be used to select for and identify immunoglobulins or portions of immunoglobulins having desirable binding properties.

Genes encoding peptides and proteins selected and identified using the methods of the present invention, including immunoglobulins and fragments thereof, can be subcloned and expressed in any of a number of cell types, or in the case of peptides, chemically synthesized, and produced for use in research, commercial, industrial, therapeutic, or diagnostic purposes.

### BRIEF DESCRIPTION OF THE FIGURES

FIG. 1 depicts a scheme for integrating genes encoding M13 phage packaging functions into the E. coli chromosome. a) map of the E. coli chromosome (100 minute system) showing the five att sites for integration of lamdoid phages. b) linear map of an integrating plasmid having a regulatable promoter (P(x)), multiple cloning site (MCS), an attachment site (att), a conditional origin of replication (ori_{R}), an antibiotic resistance gene (ant^{R}) and transcription terminators (t_{L3}, t₁t₂). c) an example of modules that can be integrated into E. coli chromosomal att sites.

FIG. 2 shows a preferred aspect of the present invention. a) a host strain chromosome having integrated modules 1-4, as shown in FIG. 1c. b) a phage display expression construct (PDEC) having a gene encoding a fusion protein comprising immunoglobulin light chain (LC) fused to M13 cpIII, and an immunoglobulin heavy chain (HC) gene.

FIG. 3 depicts a method for identifying an antibody that binds a substance of interest using the methods of the present invention. 1) transformation of a bacterial strain of the present invention having modules 1-4 (M1, M2, M3, M4) integrated into the chromosome with a phagemid phage display expression construct (PDEC), 2) induction of the strain to produce phage particles, 3) panning of phage particles against an antigen of interest, 4) infection of a bacterial strain of the present invention with selected phage, and 6) isolation of phagemid DNA from bacteria.

FIG. 4 is a diagram of the four modules of M13 genes integrated into host BW28357/AV100, a packaging strain of the present invention, showing the promoters and phage packaging function genes of the modules.

FIG. 5 is a map of the chromosome of BW28357/AV100, a packaging strain of the present invention, showing the integration sites of four modules shown in FIG. 4.

FIG. 6 depicts the results of one experiment, which shows ethidum bromide stained gels showing the results of packaging the pComb3 plasmid with packaging strain BW28357/AV100. The strain was transformed with pComb3, and then individual colonies were grown up and induced for M13 phage production. The bacterial media was subjected to phage DNA isolation procedures and PCR with pComb3 specific primers. a) negative control using isolation and PCR procedures on BW28357/AV100 that was not transformed with phagemid pComb3 showing no detectable products; b) positive PCR control using isolated phagemid pComb3 as a template showing products of the expected size; and c) PCR after phagemid isolation using BW28357/AV100 transformed with phagemid pComb3, showing packaging products of the expected size (each lane shows the results from an independent colony for c)).

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the laboratory procedures in chemistry, microbiology, molecular biology, cell biology, and cell culture described below are well known and commonly employed in the art. Conventional methods are used for these procedures, such as those provided in the art and various general references (Kay, Winter, and McCafferty, eds., Phage Display of Peptides and Proteins, A Laboratory Manual, Academic Press: San Diego (1996); C. F. Barbas III, D. R. Burton, J. K. Scott, and G. J. Silverman, Phage Display. A Laboratory Manual, Cold Spring Harbor, N.Y. (2001); Sambrook et al., Molecular Cloning: A Laboratory Manual, 2nd edition, Cold Spring Harbor Press, Cold Spring Harbor, N.Y. (1989); Ausubel et al., Current Protocols in Molecular Biology, John Wiley and Sons (1998); and Harlow and Lane, Antibodies, A Laboratory Manual, Cold Spring Harbor Press (1988), all herein incorporated by reference in their entireties). Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

A “filamentous phage” or “filamentous bacteriophage” is a bacteriophage (a type of virus that infects bacteria) comprising a single-stranded, circular DNA molecule that is encased by proteins to form a rod shaped fiber. Examples of filamentous phage are M13, fl, fd, ZJ/2, Ec9, AE2, and delta A, which infect E. coli; Pf1, Pf2, and Pf3, which infect Pseudomonas aeruginosa; Xf, which infects Xanthomonas oryzae; and v6, which infects Vibrio parahaemolyticus.

The term “phage particle” refers to the structure in which the full complement of filamentous phage coat proteins are assembled around a nucleic acid molecule in essentially the same way as is found in the wild-type filamentous phage. Preferably, the phage particle is infectious, but this need not be the case.

An “F plasmid” is a plasmid harbored by bacteria that encodes the structural proteins of the pilus that enables bacterial conjugation and infection by filamentous phage.

“Stably comprising” means that a host cell contains a gene either integrated into the chromosome, or on an autonomously replicating plasmid that can be maintained in the host cells of the population by selection.

A “phage coat protein” is one of several proteins that encase the nucleic acid of a phage particle. The f series of filamentous phage that infect E. coli (M13, fd, and fl) have five coat proteins, known as cpIII, cpIV, cpVI, cpVIII, and cpIX.

A “fusion protein” is a protein that is synthesized from an open reading frame that is the result of fusing the open reading frames that encode two (or more) proteins, or one ore more peptides and one or more proteins.

A “chimeric protein” is a protein that is synthesized from an open reading frame that is the result of fusing the open reading frames that encode portions of two (or more) proteins, or one or more peptides and a portion of one or more proteins.

An “infectious filamentous phage particle” is a phage particle that can infect a bacterium. In E. coli, infection occurs through the sex pilus (F pilus), encoded by the tra genes on the F plasmid.

A “helper phage” is a phage that supplies genes that encode packaging functions for a filamentous phage. A construct that has packaging sequences, but does not have all of the genes necessary for filamentous phage packaging, can be packaged into a phage particle when a helper phage is introduced into the same strain as the construct.

As used herein, a “packaging function” is a protein that is encoded on a wild-type filamentous phage genome that promotes the packaging of a filamentous phage. Packaging functions can be proteins that form part of the phage coat, proteins that function in the replication of the phage genome (or any nucleic acid with appropriate phage replication sequences), proteins that function in the assembly of the phage particle, including proteins that bind the nucleic acid prior to packaging and proteins that form pores in the bacterial host membrane for phage extrusion.

A “phagemid” is a plasmid that can replicate as a plasmid in bacteria (has a plasmid origin of replication) and also has a filamentous phage origin of replication and filamentous phage packaging sequences, such that under appropriate conditions it can be produced in single-stranded DNA form and packaged as a phage by the bacteria that harbor it.

As used herein, a “phage display expression vector” is a vector that comprises at least one open reading frame that encodes at least a portion of a filamentous phage coat protein, and a cloning site that occurs within or at one end of the open reading frame, such that a sequence encoding a peptide or protein can be fused with the coat protein open reading frame to encode a fusion protein or chimeric protein. A phage display expression vector also comprises filamentous phage packaging sequences, and a filamentous phage origin of replication, and preferably also includes a selectable marker and a plasmid origin of replication. A phage display expression vector can also include other sequences that are linked in frame with the coat protein encoding sequence, such as, but not limited to, sequences that encode flexible peptide linkers, sequences that encode protease or peptidase recognition sites, sequences that encode protein splicing sequences, or sequences that encode epitopes that can be used as “tags” to purify or detect a peptide or protein (e.g., the myc, FLAG, or hemaglutinin tags, a plurality of histidine residues, etc.). A phage display expression vector can also include open reading frames that encode other proteins, including, but not limited to, phage packaging functions, prokaryotic polymerases, regulatory molecules, immunoglobulin chains, receptors, or enzymes, or subunits or portions of any of these.

As used herein, a “phage display construct” is a phage display expression vector that comprises a nucleic acid sequence of interest, random sequence, or sequence from a library, such that the sequence of interest, random sequence, or sequence from a library can form a fusion protein or chimeric protein with an open reading frame that encodes at least a portion of a coat protein.

A “display peptide” is a peptide that is fused to at least a portion of a coat protein of a filamentous phage. Display peptides can be generated by cloning sequences encoding the peptide to be displayed into a phage display expression vector such that the open reading frame of the peptide is fused to the open reading frame of the coat protein or portion thereof.

As used herein, a “peptide linker” or “linker” is a peptide sequence that joins two peptide or protein sequences. Preferably, a linker provides spacing between the peptides or proteins such that they are able to retain their biological or biochemical activity and function in their intended manner. For example, a linker can comprise a flexible peptide that separates a display protein or peptide from a coat protein of a filamentous phage. In this way the display protein or peptide moiety of a fusion or chimeric coat protein can be positioned at some distance from the coat protein moiety, such that, for example, the display peptide or protein does not interfere with a domain of the coat protein that may mediate infectivity or assembly with the phage particle. Use of a linker between a display protein or peptide and a coat protein can also promote proper folding of a display protein or peptide when in the context of a fusion or chimeric protein. Linkers can be chosen and designed based on such properties as, for example, their length and their flexibility, or lack of stable secondary structure. Linkers can have other properties as well, for example, they can comprise protease recognition sites that allow cleavage of the display peptide moiety from the coat protein moiety of a fusion or chimeric protein. Nonlimiting examples of linkers that can be useful in the present invention include, for example, peptide sequences that comprise hydrophilic amino acid residues and amino acid residues with short side chains, including those having with glycine, serine, and proline residues (see, for example Dubel et al. Gene 128: 97-101 (1993); Barbas et al. Proc. Natl. Acad. Sci. 88: 797807982 (1991); U.S. Pat. No. 5,258,498 issued Nov. 2, 1993 to Huston et al. and U.S. Pat. No. 5,908,626 issued Jun. 1, 1999 to Chang et al., all herein incorporated by reference).

A “display protein” is a protein that is fused to at least a portion of a coat protein of a filamentous phage. Display proteins can be generated by cloning sequences encoding the protein to be displayed into a phage display expression vector such that the open reading frame of the protein is fused to the open reading frame of the coat protein or portion thereof.

A “secretory leader peptide” or “signal sequence” (also called a “leader peptide”, “leader sequence”, “secretory sequence”, “secretory peptide”, “signal peptide”, or similar terms) is an amino acid sequence typically positioned at the amino end of a polypeptide, that carries or directs the transport of the polypeptide through the inner membrane into the periplasmic space, and possibly outside the cell wall into the extracellular milieu.

“Isolated polynucleotide” refers to a polynucleotide of genomic, cDNA, or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

“Isolated protein” refers to a protein of cDNA, RNA derived from cDNA, DNA, RNA or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source (for example, free of cellular proteins), or (4) is expressed by a cell from a different species, or (5) does not occur in nature.

“Peptide” is a sequence of two or more amino acids joined by peptide bonds. Peptides can include other moieties, such as chemical groups, drug molecules, detectable labels, or specific binding members that are reversibly or irreversibly bound to one or more amino acids of the peptide.

“Polypeptide” is used herein as a generic term to refer to protein or fragments or analogs of a protein.

“Active fragment” refers to a fragment of a parent molecule, such as an organic molecule, nucleic acid molecule, or protein or polypeptide, or combinations thereof, that retains at least one activity of the parent molecule.

“Naturally occurring” refers to the fact that an object can be found in nature. For example, a polypeptide or polynucleotide sequence that is present in an organism, including viruses, that can be isolated from a source in nature and that has not been intentionally modified by man in the laboratory is naturally occurring.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refers to polynucleotide sequences that effect the expression of coding and non-coding sequences to which they are operably linked. When the control sequences are used to control the transcription of DNA template, it is also called a transcription regulatory region. The nature of such control sequences differs depending upon the host organisms and enzymes; in prokaryotes, such control sequences generally include promoter, ribosomal binding site, and translation initiation and termination codons; in eukaryotes, generally, such control sequences include promoters, optionally enhancers, and translation initiation and termination sequences. The term control sequences is intended to include components whose presence can influence expression, and can also include additional components whose presence is advantageous, for example, leader sequences and fusion partner sequences.

A “transcription regulatory region” is a region of a nucleic acid that controls the transcription of a nucleic acid sequence to which it is operably linked.

A “ribosome binding site” or “ribosome binding sequence” is a nucleotide sequence that allows the binding of the ribosome to a nucleic acid molecule. Ribosome binding sites known in the art that allow for ribosome binding and the initiation of translation are, for example, Shine-Dalgarno sequences, Kozak sequences, and IRES sequences. As used herein, Shine-Dalgorno sequences and Kozak sequences can be identified canonical sequences, or substantially homologous sequences that can be bound by ribosomes and thereby initiate translation. IRES sequences can be those already identified, or any identified in the future, such as by functional assay. A “ribosome RNA binding sequence” specifies that the nucleotide sequence consists of or essentially consists of an RNA sequence.

“Nucleic acid” or “nucleic acid molecule” or “polynucleotide” refers to a polymeric form of nucleotides of a least six bases in length. A nucleic acid molecule can be DNA, RNA, or a combination of both. A nucleic acid molecule can also include sugars other than ribose and deoxyribose incorporated into the backbone, and thus can be other than DNA or RNA. A nucleic acid can comprise nucleobases that are naturally occurring or that do not occur in nature, such as xanthine, derivatives of nucleobases such as 2-aminoadenine and the like. A nucleic acid molecule of the present invention can have linkages other than phosphodiester linkages. A nucleic acid molecule can also be a peptide nucleic acid molecule (PNA) or can comprise PNA residues. A nucleic acid molecule can be of any length, and can be single-stranded or double-stranded, or partially single-stranded and partially double-stranded. A nucleic acid molecule can comprise other entities, such as drug molecules, detectable labels, linking moieties, or specific binding members.

“Directly” in the context of a biological process or processes, refers to direct causation of a process that does not require intermediate steps, usually caused by one molecule contacting or binding to another molecule (the same type or different type of molecule). For example, molecule A contacts molecule B, which can cause molecule B to exert effect X that is part of a biological process. In terms of binding, “directly” means that molecule A contacts and binds molecule B without intermediate molecules that mediate the binding.

“Indirectly” in the context of a biological process or processes, refers to indirect causation that requires intermediate steps, usually caused by two or more direct steps. For example, molecule A contacts molecule B to exert effect X which in turn causes effect Y. In terms of binding, “indirectly” means that molecule A binds molecule B by contacting at least one intermediate molecule that mediates the binding.

“Sequence homology” refers to the proportion of base matches between two nucleic acid sequences or the proportion of amino acid matches between two amino acid sequences. When sequence homology is expressed as a percentage, for example 50%, the percentage denotes the proportion of matches of the length of sequences from a desired sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and most preferably not less than 19 matches out of 20 possible base pair matches (95%).

“Corresponds to” refers to a polynucleotide sequence that is homologous (for example is identical, not strictly evolutionarily related) to all or a portion of a reference polynucleotide sequence, or to a polypeptide sequence that is identical to all or a portion of a reference polypeptide sequence. In contradistinction, the term “complementary to” is used herein to mean that the complementary sequence will base pair with all or a portion of a reference polynucleotide sequence. For illustration, the nucleotide sequence TATAC corresponds to a reference sequence TATAC and is complementary to a reference sequence GTATA.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; a group of amino acids having acidic side chains is aspartic acid and glutamic acid; and a group of amino acids having sulfur-containing side chan is cysteine and methionine. Preferred conservative amino acid substitution groups are: valine-leucine-isoleucine; phenylalanine-tyrosine; lysine-arginine; alanine-valine; glutamic acid-aspartic acid; and asparagine-glutamine.

“Test compound” refers to a chemical, compound, composition or extract to be tested by at least one method of the present invention for at least one activity for at least one activity such as putative modulation of a biological process or specific binding capability. Test compounds can include small molecules, drugs, proteins or peptides or active fragments thereof, such as antibodies or fragments or active fragments thereof, nucleic acid molecules such as DNA, RNA or combinations thereof, antisense molecules or ribozymes, or other organic or inorganic molecules, such as lipids, carbohydrates, or any combinations thereof. Test compounds that include nucleic acid molecules can be provided in a vector, such as a viral vector, such as a retrovirus, adenovirus or adeno-associated virus, a liposome, a plasmid or with a lipofection agent. Test compounds, once identified, can be agonists, antagonists, partial agonists or inverse agonists of a target. A test compound is usually not known to bind to the target of interest.

“Control test compound” refers to a compound known to bind to the target (for example, a known agonist, antagonist, partial agonist or inverse agonist). Test compound does not typically include a compound added to a mixture as a control condition that alters the function of the target to determine signal specificity in an assay. Such control compounds or conditions include chemicals that (1) non-specifically or substantially disrupt protein structure (for example denaturing agents such as urea or guanidinium, sulfhydryl reagents such as dithiothreitol and beta-mercaptoethanol), (2) generally inhibit cell metabolism (for example mitochondrial uncouplers) and (3) non-specifically disrupt electrostatic or hydrophobic interactions of a protein (for example, high salt concentrations or detergents at concentrations sufficient to non-specifically disrupt hydrophobic or electrostatic interactions). The term test compound also does not typically include compounds known to be unsuitable for a therapeutic use for a particular indication due to toxicity to the subject. Usually, various predetermined concentrations of test compounds are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test compounds, the concentration of test chemical used can be expressed on a weight to volume basis. Under these circumstances, the following ranges of concentrations can be used: between about 0.001 micrograms/ml and about 1 milligram/ml, preferably between about 0.01 micrograms/ml and about 100 micrograms/ml, and more preferably between about 0.1 micrograms/ml and about 10 micrograms/ml.

“Target” refers to a biochemical entity involved in a biological process. Targets are typically proteins that play a useful role in the physiology or biology of an organism. A therapeutic composition or compound typically binds to a target to alter or modulate its function. As used herein, targets can include, but not be limited to, cell surface receptors, G-proteins, G-protein coupled receptors, kinases, phosphatases, ion channels, lipases, phosholipases, nuclear receptors, transcription factors, intracellular structures, tubules, tubulin, antibodies and the like.

A “therapeutic target” or a “pharmaceutical target” is a target that when modulated can have a therapeutic effect.

A “purification target” is a target that is useful in purification schemes, such as, for example, regions of antibodies such as the Fc region.

A “diagnostic target” is a target that is useful in diagnostics, such as cell surface epitopes or markers on etiological agents.

“Label” or “labeled” refers to incorporation of a marker that may or may not be used for detection purpose. For example by incorporation of a radiolabled compound or attachment to a polypeptide of moieties such as biotin that can be detected by the binding of a section moiety, such as marked avidin. On the other hand, if a protein is labeled by a biotin, the protein can be attached to a nucleic acid that is labeled with an avidin. Thus, the protein and nucleic acid can form a complex. Various methods of labeling polypeptide, nucleic acids, carbohydrates, and other biological or organic molecules are known in the art. Such labels can have a variety of readouts, such as radioactivity, fluorescence, color, chemiluminescence or other readouts known in the art or later developed. The readouts can be based on enzymatic activity, such as beta-galactosidase, beta-lactamase, horseradish peroxidase, alkaline phosphatase, luciferase; radioisotopes (such as ³H, ¹⁴C, ³⁵S, ³²P, ³³P, ¹²⁵I or ¹³¹I); fluorescent proteins, such as green fluorescent proteins; or other fluorescent labels, such as FITC, rhodamine, and lanthanides. Where appropriate, these labels can be the product of the expression of reporter genes, as that term is understood in the art. Examples of reporter genes are beta-lactamase (U.S. Pat. No. 5,741,657 to Tsien et al., issued Apr. 21, 1998) and green fluorescent protein (U.S. Pat. No. 5,777,079 to Tsien et al., issued Jul. 7, 1998; U.S. Pat. No. 5,804,387 to Cormack et al., issued Sep. 8, 1998).

“Specific binding member” is one of two different molecules having an area on the surface or in a cavity which specifically binds to and is thereby defined as complementary with a particular spatial and polar organization of the other molecule. A specific binding member can be a member of an immunological pair such as antigen-antibody, biotin-avidin, hormone-hormone receptor, nucleic acid duplexes, IgG-protein A, DNA-DNA, DNA-RNA, and the like.

“Substantially pure” refers to an object species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, as substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods) wherein the composition consists essentially of a single macromolecular species or activity. The inventors recognize that an activity may be caused, directly or indirectly, by a single species or a plurality of species within a composition, particularly with extracts.

“Pharmaceutical agent or drug” refers to a chemical, composition or activity capable of inducing a desired therapeutic effect when property administered by an appropriate dose, regime, route of administration, time and delivery modality.

“Sample” means any biological sample, preferably derived from a test animal, such as a mouse, rat, rabbit or monkey, or a patient, such as a human. Samples can be from any tissue or fluid, such as neural tissues, central nervous tissues, internal organs such as pancreas, liver, lung, kidney, muscle, skeletal muscle, urine, feces, blood, fluids from body cavities or the central nervous system, or samples from various body cavities such as the mouth or nose. Samples derived from urine and feces contain cells of the immunological, urinary or digestive tract and can be a rich source of sample. Such samples can be obtained using methods known in the art, such as biopsies, aspirations, scrapings or simple collection. A sample can be taken from a test animal or patient that is either living or dead.

A “DNA” molecule refers to either single- or double-stranded deoxyribonucleic acid. A DNA molecule can include nucleotide analogues or derivatives, such as dideoxynucleotides, or nucleotides comprising non-naturally occurring bases, such as inosine, and can comprise one or more linkages other than phosphodiester linkages, such as for example, phosphoramide or phosphothioate linkages. A DNA molecule can also comprise other chemical moieties, such as labels, specific binding members, and linking moieties.

A “transcription termination sequence” or “termination sequence” refers to a region or structure of a nucleic acid molecule that impedes with the progress of, stalls, or stops the functional migration of an RNA polymerase along the nucleic acid template.

A “random sequence” refers to a fully random, partially random, or semi-random sequence of nucleic acid bases that forms a nucleic acid molecule or amino acids that form a polypeptide. Random sequences can be made using synthetic methods as they are known in the art, such as solid phase nucleic acid or solid phase polypeptide synthesis, or by enzymatic methods, such as polymerase reactions or digesting polypeptides or nucleic acids of natural or synthetic origin to obtain fragments thereof, or by any combination of these methods. Fully random refers to 1) sequences that have been made without statistical weight to the probability of inserting any one of the set of naturally-occurring bases or amino acids at a given position of the random sequence, or 2) sequences that have been made by fragmentation of at least one nucleic acid molecule. Semi-random refers to sequences that have been made with statistical weight as bases/amino acids and/or their sequence and can be made using synthetic methods known in the art or by digesting polypeptides or nucleic acid molecules (see, U.S. Pat. No. 5,270,163 to Gold et al., issued Dec. 14, 1993; and U.S. Pat. No. 5,747,253 to Ecker et al., issued May 5, 1998). Semi-random sequences can be nucleic acid or amino acid sequences that have been synthesized such that particular sequence combinations are preferred over other sequence combinations. For example, a semi-random nucleic acid sequence can be biased to preferentially include only a subset of the nucleic acid codons that encode particular amino acids, or can be biased such that the frequency of stop codons in the sequence is reduced. Similarly, a semi-random nucleic acid sequence can be synthesized such that, for example, codons for hydrophobic amino acids are less abundant in the sequence than would occur if the sequence were totally random. Semi-random sequences can be made by directed chemical synthesis, and can, for example, be based on the synthesis of preferred codons that can be built into a multi-codon sequence as disclosed in PCT application US99/22436 (WO 00/18778) to Lohse et al., published Apr. 6, 2000, which is herein incorporated by reference. Partially random sequences are sequences that are in part known or identified sequences and are in part fully random or partially random sequences, and can also be made by modifying or adding to identified or fixed sequences (Pasqualini and Ruoslahti, Nature 380:364-366 (1999); and U.S. Pat. No. 5,270,163 to Gold et al., issued Dec. 14, 1993).

A “sequence of interest” refers to a nucleic acid sequence or nucleic acid molecule that has been or can be selected for by screening or otherwise identified. A sequence of interest can also be at least a portion of a known nucleic acid molecule or nucleic acid sequence. Preferably, an activity, such as an enzymatic activity or binding activity, of the amino acid sequence that can be partially or entirely encoded by the sequence of interest is known (but that need not be the case), and the sequence of interest includes sequences encoding at least one such activity or a portion of such activity.

“Substance of interest” refers to a compound that has been selected for screening peptides or complexes of the present invention, or has been identified using the methods of the present invention. A substance of interest can be an organic or inorganic molecule, including a peptide, protein, carbohydrate, lipid, steroid, nucleic acid, polymer, small molecule, metal, or any combination of these (for example, a lipoprotein), and can be a complex (a ribosome, multimeric enzyme, complex polymer), organelle, virus, or cell. A substance of interest can be provided in soluble or insoluble form, on a surface (including a metallic, ceramic, glass, paper, polymeric, viral, or cellular surface) or as a part of a polymer, including a gel.

“Solid support” refers to any solid support that can be used in a method of the present invention. Preferably, a solid support is used to immobilize a nucleic acid molecule of the present invention or a complex of the present invention. In addition, a solid support can be used to immobilize a substance of interest, a cell, an etiological agent, or other moiety. Solid substrates can take any form, such as sheets, membranes (such as nitrocellulose or nylon), polymeric surfaces, including wells (such as microtiter wells), beads, or chips, such as glass, nylon, or silica sheets that comprise arrays of nucleic acids, proteins, or other molecules. Solid supports can be of any appropriate material, such as paper, polymers, metals, glass, or silica and can be magnetic in nature. Preferred solid substrates include polystyrene, polycarbonate, latex, polyacrylamide, sepharose, nylon, nitrocellulose, glass, silica, and magnetite.

“On or within a cell” refers to a moiety, such as a receptor or biomolecule that resides on the surface of a cell, within the outer membrane of a cell, or within a cell. Within a cell refers to any locus within a cell, such as in the cytoplasm or within or associated with an organelle, such as, for example, a mitochondria, nucleus or Golgi apparatus.

A “cell” refers to any cell, such as a cell of prokaryotic (such as bacterial) or eukaryotic origin. Eukaryotic cells include, for example, single cell organisms such as yeast and multicellular organisms such as invertebrates, plants and vertebrates. Invertebrates include parasites such as worms and vertebrates include cold-blooded organisms (such as reptiles and amphibians) and warm-blood organisms, such as mammals, including humans. A cell can be part of a sample of tissue, fluid or organ of a multicellular organism, or can be part of a multicellular organism itself.

“In vitro” refers to procedures that are performed outside of a cell. For example, purified enzymes or extracts of cells can be used to perform procedures in a vessel, such as a test tube.

“Ex vivo” refers to procedures that are performed outside of a multicellular organism, but use whole cells. For example, live cells from a subject, such as a human, can be cultured outside of the body and these cells can be used in testing procedures.

“In vivo” refers to procedures that are performed on a whole organism, such as a subject, including a human, such as in clinical trials. In vivo procedures can also be performed on non-human subjects, such as animal models.

A “normal cell” refers to a cell whose processes and characteristics are in conformance with an average cell of that type. For example, a normal lung cell does not exhibit the proliferation and metastatic capabilities of a cancerous lung cell.

An “abnormal cell” refers to a cell whose processes and characteristics are not in conformance with an average cell of that type. For example, a CD4+ cell infected with a virus, such as HIV, does not exhibit the lifespan of a normal CD4+ cell.

A “neoplastic cell” refers to a cell that exhibits the processes and characteristics of a neoplasm, such as tumors, cancers, carcinomas and the like.

A “virus infected cell” refers to a cell that has been infected with a viable virus and exhibits or will exhibit characteristics of that infection.

An “etiological agent” refers to any etiological agent, such as bacteria, parasites, fungi, viruses, prions and the like.

A “library” refers to a group of two or more nucleic acid molecules. The members of a library can be mixed into a single population, such as in a single container. Alternatively, the members of a library can be provided separately in different containers, such as in the wells of microtiter plates or separate containers in a larger container, such as vials in a box.

Other technical terms used herein have their ordinary meaning in the art that they are used, as exemplified by a variety of technical dictionaries, such as the McGraw-Hill Dictionary of Chemical Terms and the Stedman's Medical Dictionary.

Introduction

The present invention recognizes the need to identify peptides and proteins that have desirable properties, such as binding and catalytic properties, that can be used as reagents, research tools, and as therapeutic and diagnostic compounds. The selection and identification procedure for peptides and proteins and the nucleic acids that encode them known as “phage display” has provided a means for achieving these aims. However, inefficiencies in phage display as it is currently practiced can prolong the screening procedure and lead to less than optimal results. The present invention provides compositions and methods for increasing the efficiency of phage display, and provides other benefits as well.

A first aspect of the present invention is a bacterial strain that stably comprises at least one gene that encodes a protein having a packaging function for a filamentous phage. A bacterial strain of the present invention can be transformed with a phage display expression construct that comprises a coat protein fusion protein or coat protein chimeric protein and used to generate packaged recombinant phage particles in the absence of a helper phage. In some preferred aspects of the invention, the bacterial strain contains nine of the ten genes of an Ff filamentous phage that encode packaging functions, where a filamentous phage gene not contained by the bacterial strain is a coat protein gene that is provided on a phage display expression construct for the generation of fusion coat proteins or chimeric coat proteins.

The present invention also includes methods of using a bacterial strain of the present invention to generate recombinant filamentous phage without the use of a helper phage. A recombinant filamentous phage generated by the methods of the present invention preferably includes a phage display expression construct that encodes at least one coat protein fusion protein or coat protein chimeric protein, and includes at least one coat protein fusion protein or coat protein chimeric protein as a part of the protein coat that encases the nucleic acid construct. Recombinant filamentous phage generated by the methods of the present invention that express display peptides with desirable properties can be selected for, and the display peptide can be identified by isolating and sequencing the phage display expression construct of the selected phage using methods known in the art. Preferred methods of the present invention include those that select for heavy chain and light chain antibody molecules with affinity for substances of interest.

The present invention also includes peptides and proteins identified using the methods of the present invention. Such peptides and proteins can be generated from random sequences, can be generated from nucleic acid molecules from libraries (such as cDNA or genomic libraries), or can be any sequences of interest. The peptides or proteins of interest can be generated by mutating known nucleic acid sequences, random nucleic acid sequences, or nucleic acid sequences from libraries, or portions thereof. Peptides or proteins selected for using the methods of the present invention can be antibodies, ligands, or receptors, or can be enzymes, hormones, extracellular matrix components, structural proteins, or any peptides or proteins of interest, including any portion or portions thereof or combinations of proteins and peptides. Preferred peptides and proteins of the present invention include immunoglobulin heavy and light chains, and fragments thereof. Peptides or proteins selected f6r and identified using the methods of the present invention can be used for a variety of purposes, including research applications and therapeutic or diagnostic applications.

The inventors also envision that aspects and embodiments of the invention described herein can also be combined to make additional embodiments and aspects of the present invention.

I. Bacterial Strains Stably Comprising Genes Encoding Packaging Functions for Use in Phage Display

A first aspect of the present invention is a bacterial strain that stably comprises at least one gene that encodes a protein having a packaging function of a filamentous bacteriophage, and that does not require infection with a helper phage to package a phage display expression construct. For the purposes of the present invention, a filamentous bacteriophage is any filamentous phage that can infect a bacterial strain, including, but not limited to, M13, fl, fd, ZJ/2, Ec9, AE2, and delta A, which infect E. coli; Pf1, Pf2, and Pf3, which infect Pseudomonas aeruginosa; Xf, which infects Xanthomonas oryzae; and v6, which infects Vibrio parahaemolyticus. The M13, fl, and fd phages, a group of highly homologous phages that infect E. coli and are collectively known as the Ff phages, are particularly preferred filamentous bacteriophages of the present invention. A packaging function of a filamentous bacteriophage is any function encoded by the wild-type phage genome that effects or promotes the replication of the phage genome, the assembly of the phage fiber, rod, or particle, or the extrusion of the phage genome (preferably as a rod or particle) from the bacterial host. Thus, the genomes of the Ff filamentous phage comprise eleven genes that encode packaging functions, including the replication of the phage DNA (genes II and X), the assembly of the phage fiber (genes III, V, VI, VII, VIII, and IX), and the formation of membrane pores (genes I, IV, and XI) that allow the release of the packaged phage from the bacterial host.

A requirement of the bacterial host strain is that it is infectable by filamentous phage, and therefore, for the bacterial strains that are preferred, the bacterial strains will have the F factor that encodes the pilus through which filamentous phage infection occurs. The F factor can be on an F plasmid; many strains of E. coli are available in which the F factor is maintained by selection of the F plasmid using, for example, nutritional markers.

However, host strains of the present invention can also be strains other than those that naturally harbor an F factor. In some aspects of the present invention, F factors can be transmitted to strains that would not otherwise carry them. In other aspects of the present invention, it can be possible to use strains that can take up exogenous DNA, such as phagemid DNA, without the requirement of the phage infection process (and therefore the pilus encoded by the F factor). Of particular interest are the so-called “naturally transformable” bacterial strains such as for example, certain Haemophilus, Pseudomonas,Vibrio, Helicobacter, and Streptococcus strains (Frischer et al., Curr Microbiol 33: 287-291 (1996); Frischer et al., Appl. Environ Microbiol 56: 3439-3944 (1990); Sikorski et al., Appl. Environ Microbiol. 68: 865-873 (2002); Saunders et al. Microbiol. 145: 3523-3528 (1999); Mercer et al. FEMS Microbiol. Lett. 179: 485-490 (1999); Stewart and Sinigalliano, AntonieVan Leeuwenhoek 59: 19-25 (1991)). Such strains, which do not require special treatment (such as electroporation, calcium chloride, etc.) for their uptake of exogenous DNA, may be of particular advantage where libraries are to be transformed into a bacterial strain for phage display.

As used herein, a “helper phage” is a phage that supplies genes that encode packaging functions for a filamentous phage. A construct that has packaging sequences, but does not have all of the genes necessary for filamentous phage packaging, can be packaged into a phage particle when a helper phage is introduced into the same strain as the construct. The bacterial strains of the present invention do not require the presence of a helper phage for the packaging of a phage display expression vector construct.

As used herein, a “phage display expression construct” is a construct that comprises at least one open reading frame that encodes at least a portion of a filamentous phage coat protein, and a cloning site that occurs within or at one end of the open reading frame, such that a sequence encoding a peptide or protein can be fused with the coat protein open reading frame to encode a fusion protein or chimeric protein. A phage display expression construct also comprises a phage origin of replication, and preferably also includes a selectable marker and a plasmid origin of replication.

A bacterial strain of the present invention can stably comprise from one to the full complement of genes that encode filamentous phage packaging functions. A bacterial strain of the present invention can stably comprise a single copy or multiple copies of a particular gene that encodes a filamentous phage packaging functions. Preferably, a bacterial strain of the present invention stably comprises a plurality of genes that encode filamentous phage packaging functions. In some preferred aspects of the present invention, a bacterial strain of the present invention stably comprises all but one of the genes that encode packaging functions of an Ff phage, and the gene that encodes a packaging function that is not stably comprised by the bacterial strain is a coat protein. Preferably, in these embodiments the coat protein gene that is not stably comprised by the bacterial strain is encoded by gene III, gene VIII, gene VII, gene IX, or gene VI, but is more preferably a coat protein encoded by gene III, gene VIII, gene VII, or gene IX, even more preferably a coat protein encoded by gene III or gene VIII, and most preferably a coat protein encoded by gene III. Thus, in one preferred aspect of the invention, a bacterial strain is an E. coli strain that stably comprises genes I, II, IV, V, VI, VII, VIII, IX, and X of an M13, fd, or fl phage, although other aspects are also contemplated, some of which are described below.

Preferably, at least a portion of at least one coat protein gene that is not encoded by the gene or genes stably comprised by a bacterial strain of the present invention can be provided on a vector, such that a random sequence, member of a library, or sequence of interest can be cloned into the vector to generate a fusion or chimeric protein with the coat protein (a “phage display expression construct”). Thus, the gene or genes that encode filamentous phage packaging functions that are stably comprised by a bacterial strain of the present invention can, together with at least one other gene that resides on a phage display expression construct and that encodes a phage coat fusion protein or a phage coat chimeric protein, encode products that produce and package the construct in an infectious filamentous phage particle. Thus, a bacterial strain transformed with the phage display expression construct can produce phage particles that comprise coat proteins that are fusion or chimeric proteins comprising random sequences, library sequences, or sequences of interest, without the bacterial strain being infected or transfected with a helper phage.

In some embodiments, a bacterial strain of the present invention can stably comprise fewer than ten packaging function genes of an Ff filamentous phage. In these embodiments, a phage display expression vector can supply, in addition to a sequence encoding a coat protein fusion or chimeric protein, additional packaging function genes, such that a bacterial strain of the present invention transformed with a phage display expression vector designed therefore can have a complement of ten Ff phage packaging function genes, as well as sequence encoding at least a portion of the eleventh Ff packaging gene in the context of a coat protein fusion or chimeric construct. As one example, the phage display expression vector can comprise genes I, IV, and XI of an Ff filamentous phage that encode the pore-forming functions, in addition to comprising a sequence encoding a gene III coat protein fusion or chimeric protein.

In some embodiments, a bacterial strain of the present invention can stably comprise all of the packaging function genes of an Ff filamentous phage. In these embodiments, the bacterial strain can package any phagemid (or any DNA sequence having appropriate packaging sequences) in the absence of a helper phage regardless of whether a phagemid or DNA molecule having packaging sequences also encodes a packaging function or a portion thereof. For example, a bacterial strain of the present invention can stably comprises all eleven Ff packaging genes, such as but not limited to M13 genes I-XI. Optionally but preferably, a phagemid used in the methods of the present invention that is packaged by a bacterial strain of the present invention that comprises all of the packaging function genes of an Ff filamentous phage comprises at least one coat fusion protein or coat chimeric protein. The one or more coat fusion proteins or coat chimeric proteins encoded by the plasmid can be incorporated into the coat of the phage produced by the bacterial strain of the present invention along with the coat proteins encoded by genes stably comprised by the bacterial strain.

By “stably comprising” is meant that the bacterial strain retains the gene or genes encoding packaging functions either incorporated into its chromosome, or on an episome that is maintained in the strain through selection (e.g., a nutritional or drug resistance marker). A gene stably comprised by the bacterial strain can be on an F plasmid, can be on a plasmid that is compatible with a phage display expression vector, or, preferably, is integrated into the bacterial host chromosome. It is also within the scope of the present invention to provide one or more genes that encode filamentous phage packaging functions on the phage display expression vector, providing that at least one gene that encodes a filamentous phage packaging function is stably comprised by the bacterial host, and packaging of the phage display expression construct does not require the transfection or infection of the bacterial host with a helper phage. As one example, already presented above, Ff phage genes I, IV, and XI, which encode pore-forming proteins, can be provided on a phage display construct that also comprises a coat protein fusion or chimeric gene, for example, a cpIII fusion protein gene. In this case, genes II, V, VI, VII, VIII, and X, can be integrated into the bacterial chromosome (or reside on an episome, or a combination thereof). In this way the expression of genes I, IV, and XI will not compromise the growth of the bacterial host by forming membrane pores in the absence of virus formation.

Thus, the packaging functions encoded by genes that are stably comprised by a bacterial strain of the present invention can be localized to any combination of one or more chromosomal integration sites, selectable episomes, or phage display expression vectors. The only requirements of a bacterial phage packaging strain of the present invention is that the bacterial packaging strain stably comprises at least one gene that encodes a filamentous phage packaging function, and that the packaging of phage particles by the bacterial strain does not require the addition of a helper phage.

In constructing a bacterial strain of the present invention, the principles of bacterial genetics as well as the techniques and the particular genes, sequences, regulatory elements, loci, and the like that are known in the art, such as, for example, those disclosed in Niedhardt, et al., Escherichia coli and Salmonella: Cellular and molecular biology ASM Press, Washington D.C. (1996), and references cited therein, can be used.

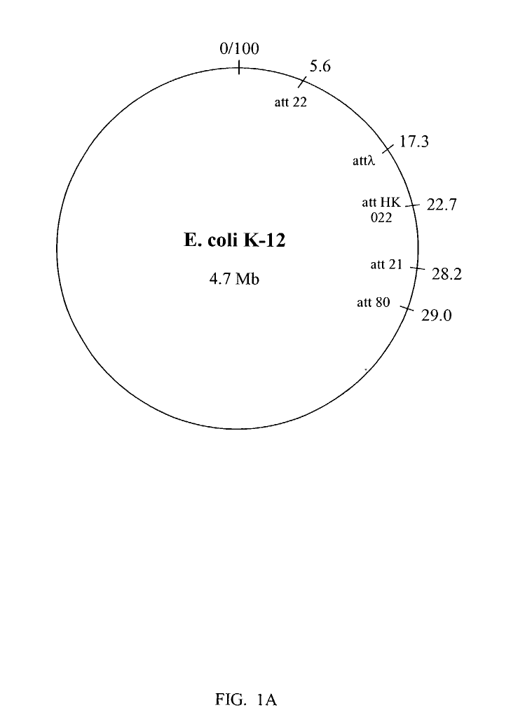

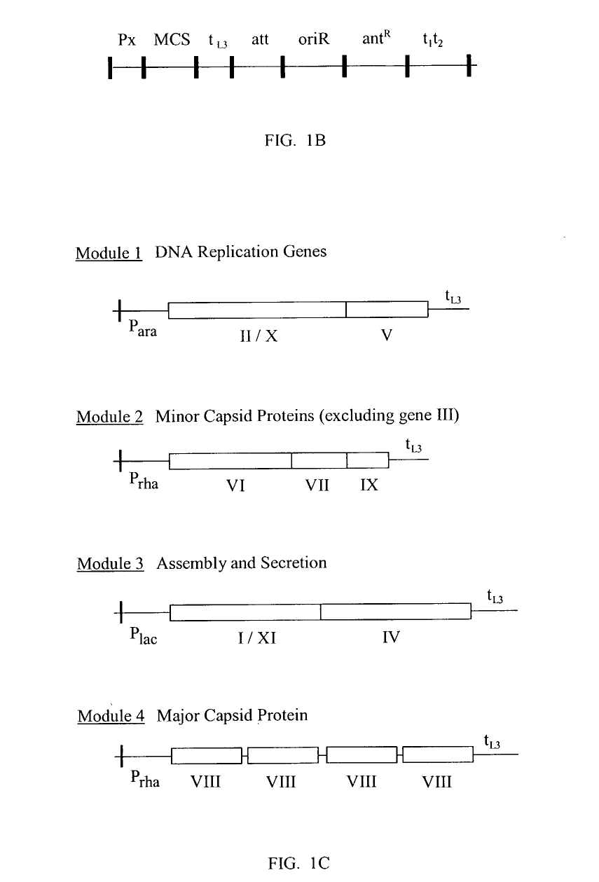

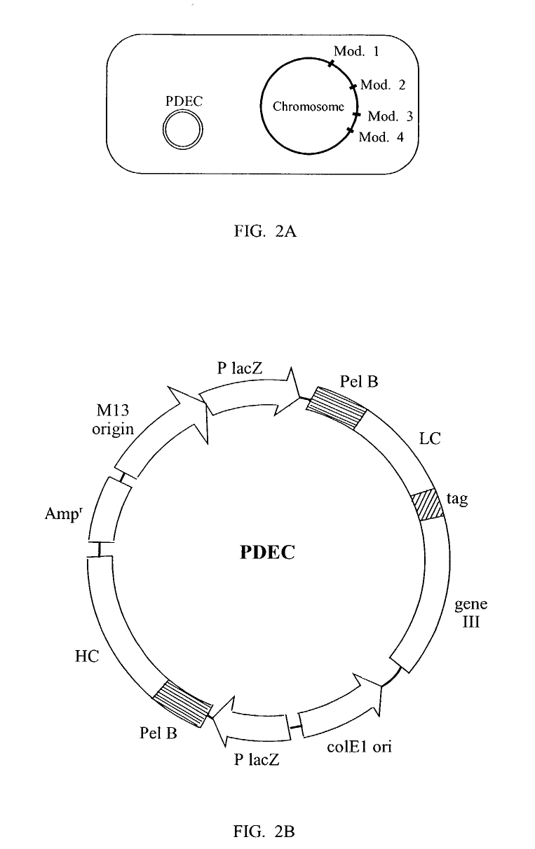

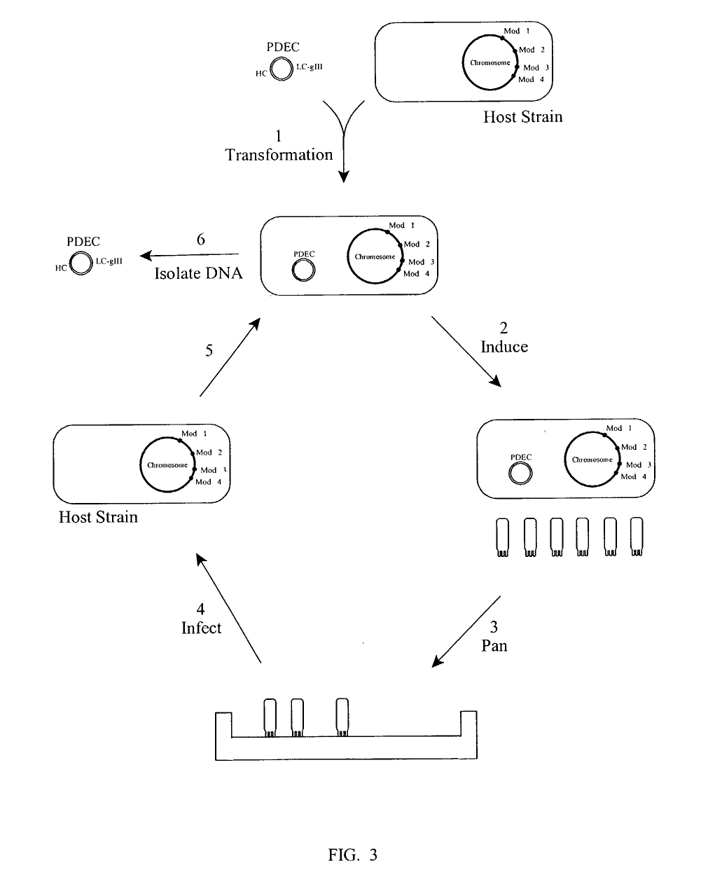

In preferred embodiments where one or more genes encoding filamentous phage packaging functions are integrated into the host chromosome, they are preferably situated at a locus where they do not interrupt critical bacterial functions. Preferred sites for the integration of packaging genes are att sites, the integration sites for lambda phage, although bacterial strains that comprise packaging function genes at other sites on the bacterial chromosome are also within the scope of the present invention. Constructs for the integration of exogenous genes at attB sites are known in the art (Diederich et al. Plasmid 28: 14-24 (1992); Haldimann and Wanner J. Bacteriol. 183: 6384-6393(2001)). Examples of att sites for the integration of lambda phage that can be used for the integration of phage packaging function genes are the att22 site, the attHK site, the att80 site, and the att 21, as depicted on the map of the E. coli chromosome in FIG. 1a.

It is also possible to integrate genes encoding filamentous phage packaging functions into a host chromosome by homologous recombination (see, for example Haldiman et al. J. Bact. 180: 1277-1286 (1998)). Homologous recombination of constructs that comprise one or more genes encoding filamentous phage packaging functions can be directed to chromosomal sites whose function can be ablated in the host, such as for example, the rha or ara loci, and can optionally use selectable markers to detect the integration event.

Inducible prokaryotic promoters can find use in the present invention by allowing the production of packaging functions to be regulated. Many regulatable promoters are known in E. coli. Of particular interest are those that have low levels of transcription until induced or derepressed by a small molecule or altered growth conditions, for example the lac promoters, including hybrid lac promoters such as the ptac and T7/lacO promoters (derepressed by galactose and galactose analogues, such as IPTG and repressed by the lac repressor and glucose), the ara promoter (induced by arabinose and repressed by glucose), the rha promoter (induced by rhamnose and repressed by glucose), and phage promoters (that can, for example, be used with temperature sensitive repressors) (Nakamura and Inouye EMBO J. 1: 771-775 (1982); Dubendorff and Studier J. Mol. Biol. 219: 45-59 (1991); Haldiman et al. J. Bact. 180: 1277-1286; Holcroft and Egan, J. Bact. 182: 6774-6782 (2000); Diederich et al. Plasmid 28: 14-24 (1992)).

An advantage of using a combination of the lac, rha, and ara promoters are that they are induced by different molecules, and so their expression levels can be regulated independently. In addition, they can all be repressed by the same molecule, glucose, when the expression of phage proteins is not desirable.

In some aspects of the present invention, promoters of different strengths can be used to direct expression of phage packaging function genes that are stably comprised by a bacterial strain of the present invention, such that the levels of phage packaging proteins will differ according to the amounts required for packaging and extrusion of phage particles. Phage packaging function genes can be cloned into modules, where a module is a construct that comprises one or more phage packaging function genes and at least one promoter that can be directed to a single locus in a bacterial chromosome. Preferably a module also comprises at least one transcription terminator sequence. However, it not required that phage packaging function genes be organized into modules for integration into a host chromosome.

Where modules are used to develop a bacterial strain of the present invention, a single module can be used, or multiple modules can be used for the integration of phage packaging function genes into the host chromosome. For example, a single module that comprises ten phage packaging function genes and a single or multiple promoters, can be used to integrate the ten packaging genes into the host strain chromosome. Alternatively, a single module that comprises all eleven of the packaging function genes of an Ff phage can be constructed for integration. Various combinations are possible using multiple modules. For example, the set of packaging function genes to be directed to the bacterial chromosome can be arranged in two, three, four, five, or more modules. For greater efficiency in constructing the strain and optimally regulating packaging function gene expression, it is preferred that packaging function genes with similar optimal levels of expression are grouped together on the same module, and preferably regulated, at least in part, by the same promoter.

In some preferred aspects of the present invention, a single promoter directs, at least in part, the expression of all of the genes in the module. In most cases, a promoter will be engineered 5′ of one or more phage packaging function genes of a module (although this need not be the case), and the engineered promoter will affect the level of expression, at least in part, of all of the genes of the module (although this need not be the case). Thus, in preferred aspects of the invention, phage packaging genes can be grouped into modules based on the similarity of desired expression levels of the genes, so that individual modules can be regulated appropriately. It is also possible to have more than one promoter in a single module (one or more promoters of a module may, for example, originate from the filamentous phage from which the packaging genes originated) such that expression of individual phage packaging function genes of the module can be modulated to some degree.

One strategy for regulating packaging genes that are stably comprised by a bacterial strain of the present invention is to use a prokaryotic promoter that requires an exogenous polymerase such as the T7, T3, or SP6 promoters. The gene encoding the exogenous promoter, optionally under the control of an inducible promoter, can be provided on the phage display construct. Thus, transforming a bacterial packaging strain of the present invention with a phage display construct can provide the fusion protein construct as well as the polymerase gene needed to activate phage packaging functions. This strategy can optionally be used with the T7lac promoter, which combines the control by induction using galactose analogues with the requirement for the T7 polymerase (Dubendorff and Studier J. Mol. Biol. 219: 45-59 (1991)). Where inducible promoters are used, molecules used to induce promoters can be, as nonlimiting examples, antibiotics, sugars, sugar analogues, nucleotides, nucleotide analogues, or pheremones. Inducible promoters can also be regulated by environmental conditions, such as, for example, temperature or oxygen availability.

Promoters used in the methods of the present invention need not be regulatable promoters. For example, promoters used in the methods of the present invention can optionally be promoters that direct constitutive gene expression. Promoters used in the methods of the present invention can be promoters from the filamentous phage from which the packaging function genes are derived. Promoters used in the methods of the present invention can also be synthetic promoters.

It is also within the scope of the present invention to have host strains that comprise multiple copies of one or more packaging function genes. The genes can optionally be provided on a single integration module. Providing a gene in multiple copies can enhance expression levels of the gene.

Bacterial strains of the present invention can be provided in kits. In addition to a bacterial strain of the present invention, a kit can comprise vectors, such as, but not limited to, one or more phage display expression vectors, solutions, and reagents. The components of the kit can be provided in one or more containers, and the kit can also contain instructions for use.

II. Methods of Using Bacterial Strains Supplying Packaging Functions in Phage Display

The present invention includes methods of using bacterial strains that stably comprise at least one gene that encodes a packaging function of a filamentous phage to select and preferably identify peptides and proteins. These methods rely on the fundamental principle of phage display technology, which is the physical linkage of a peptide or protein to the nucleic acid sequence that encodes it, by virtue of the encoding nucleic acid sequence being packaged in a phage particle that encompasses the peptide or protein of interest in its coat. The novel methods of the present invention improve the efficiency and reliability of this linkage, and eliminate the interference by helper phage.

The methods of the present invention include: cloning a nucleic acid sequence That encodes a random sequence, a sequence of interest, or a nucleic acid sequence from a library into a phage display expression vector such that said random sequence or sequence of interest or sequence from a library and the open reading frame that encodes at least a portion of a coat protein of a filamentous bacteriophage can form at least one fusion protein or chimeric protein; transforming said phage display expression vector into a bacterial strain of the present invention; and providing conditions for the bacterial strain to produce filamentous bacteriophage.

In preferred embodiments of the present invention, the method further includes: isolating at least one filamentous bacteriophage produced by the bacterial strain of the present invention that binds or interacts with at least one substance of interest; isolating at least one nucleic acid molecule from the one or more bacteriophage that bind or interact with the substance or substances of interest; and, preferably, identifying at least one peptide or protein encoded by the phage display expression vector.

A phage display expression vector can be a filamentous phage-based vector but preferably is a phagemid vector, where a phagemid is a plasmid that has a plasmid origin of replication, a filamentous phage origin of replication, and filamentous phage packaging sequences. Preferably, a phage display expression vector used in the methods of the present invention also comprises a selectable marker, such as an antibiotic resistance gene. A phage display expression vector used in the methods of the present invention can have at least one sequence that encodes at least a portion of a filamentous phage coat protein. The sequence can encode at least a portion of any filamentous phage coat protein but preferably encodes at least a portion of a filamentous phage coat protein, preferably cpIII, cpVII, cpVIII, or cpIX, more preferably at least a portion of cpIII or cpVIII, and most preferably at least a portion of cpIII. Preferably, there is at least one cloning site within or adjacent to the sequences encoding at least a portion of a coat protein, such that a sequence from a library, a random sequence, or a sequence of interest can be cloned to generate a fusion protein or chimeric protein between the peptide or protein encoded by the sequence from a library, random sequence, or sequence of interest and the coat protein, or portion thereof.

In preferred embodiments of the present invention, phage display expression vectors encode cpIII in its entirety, and N-terminal fusions with cpIII can be made with sequences from libraries, random sequences, or sequences of interest. Methods and examples of creating N-terminal fusions with full-length cpIII are known in the art, such as, for example, those disclosed in PCT/GB91/01134 and U.S. Pat. No. 6,225,447 issued May 1, 2001 to Winter et al., both herein incorporated by reference in their entireties.

Optionally, phage display expression vectors can comprise sequences that encode flexible peptide linkers in-frame with the coat protein encoding sequences. The linker-encoding sequences can be positioned such that display peptide or protein-encoding sequences cloned into the expression vector are expressed as part of a fusion or chimeric coat protein wherein the display moiety and the coat protein moiety are separated by a peptide linker. These linkers can allow for optimal folding of the display peptide or protein or greater accessibility of the display peptide or protein to the substance of interest during selection. In cases where the coat protein is cpIII, the separation of the display moiety from the cpIII moiety by a flexible peptide linker can also potentially enhance the infectivity of the cpIII protein.

A phage display expression vector of the present invention can also include sequences that encode peptide “tags” that are preferably fused in-frame with the coat protein sequences. Epitope tags are well-known in the arts of cell biology and protein purification, and can be chosen from those known in the art, such as, for example, the c-myc, hemaglutinin, FLAG, and His tags or can be developed as a peptide sequence that binds a particular specific binding member. The tags can be used for labeling or purification of the fusion peptide or phage that displays the fusion peptide. The phage display expression vector can also include sequences that encode protease recognition sites, or protein splice sites (Mathys et al. Gene 231: 1-13 (1999)). In some embodiments of the present invention, a protease recognition site or protein splice site can separate the display peptide or protein moiety from the coat protein moiety of a fusion protein. After display of the fusion protein on phage and selection of phage, the display peptide or protein can be cleaved from the coat protein on the phage (using specific proteases or conditions that activate protein splicing). This can enhance the infectivity of the phage in amplification steps. In some embodiments of the present invention, a protease recognition site or protein splice site can be used to separate a peptide tag from a fusion protein that is displayed on a phage after the tag has been used to label or purify the fusion protein.

The present invention also contemplates other fusion and chimeric constructs, including constructs using other types of cpIII fusions, including those that do not use the entire cpIII gene (see for example, PCT/GB91/01134 WO92/01047; U.S. Pat. No. 5,667,988 issued Sep. 16, 1997 to Barbas et al.; Dubel et al. Gene 128: 97-101 (1993)) and constructs using fusions with other coat proteins. Fusions and chimeras can be with any of the filamentous phage coat proteins, taking into account the necessity of maintaining the structural association of the particular coat protein of the fusion or chimera with the phage particle and the accessibility of a fused display peptide to a substance of interest used for selection.

The appropriateness and efficiency of phage display expression constructs engineered to create fusions or chimeras with a given coat protein and a display peptide can be tested using methods well known in the art (see, for example, Kay, Winter, and McCafferty, eds., Phage Display of Peptides and Proteins, A Laboratory Manual, Academic Press: San Diego (1996); and C. F. Barbas III, D. R. Burton, J. K. Scott, and G. J. Silverman, Phage Display. A Laboratory Manual, Cold Spring Harbor, N.Y. (2001)). For example, the ability of a coat protein fusion protein to assemble into infective phage particles can be tested by inducing phage synthesis and titering the resulting supernatant with respect to control constructs that have wild-type coat proteins. The ability of a display peptide to be accessible to a substance of interest can be tested by cloning a sequence encoding a known binding peptide in the phage display expression vector such that it creates a fusion or chimeric protein with a coat protein. Phage particles produced from strains comprising the expression vector can be selected using, for example, a ligand attached to a solid support. The ability to recover phage that display the peptide can be quantitated by titering. The presence of the desired construct in the captured phage can be confirmed using, for example, such methods as PCR, hybridization, or nucleic acid sequencing.

The nucleic acid sequence that is cloned into a phage display expression vector to generate a phage display expression construct can be any nucleic acid sequence, including a sequence from a library, a random sequence (including a partially random or semi-random sequence), or a sequence of interest, for example, a sequence that encodes a ligand, an antibody, an antibody variable region, a receptor or a domain of a receptor, an enzyme, etc. The sequence can be known or unknown, and can be isolated from an organism or chemically synthesized. In many uses of the present invention, one or more regions within a particular nucleic acid sequence will be subjected to mutagenizing techniques (such as, but not limited to, PCR-based mutagenesis and randomized chemical synthesis) and a library of variants of the nucleic acid sequence is cloned into a phage display expression vector. A nucleic acid sequence that is cloned into a phage display expression vector to form a fusion or chimeric coat protein gene can be of any length. Preferably, for embodiments in which the sequence is cloned to create a fusion protein with a cpIII protein, is between about 9 and about 3600 nucleotides long, more preferably between about 15 and about 2400 nucleotides long, and most preferably between about 18 and about 1200 nucleotides long. Preferred nucleic acid sequences include those encoding immunoglobulin (Ig) heavy chains and light chains, such as, but not limited to, mouse Ig heavy and light chains, and human Ig heavy and light chains.

Methods of creating constructs that include sequences of interest in optimal configurations (for example, coding sequences in-frame with other coding sequences and coding sequences operably linked to control sequences) are well-known in the art and disclosed in many of the references cited herein.

A phage display expression vector of the present invention can comprise genes and coding regions for peptides and proteins other than filamentous phage-derived proteins and peptides. For example, a phage display expression vector of the present invention can comprise genes that can be used as selectable markers, including, but not limited to, genes that confer antibiotic resistance, nutritional prototrophy, genes that encode suppressor tRNAs, regulatory molecules, and the like. In additon to selectable markers, genes on a phage display expression vector of the present invention can encode other types of proteins or transcripts, such as, but not limited to, antisense transcripts, structural proteins, or enzymes such as polymerases. In some preferred aspects of the present invention, for example, a phage display expression vector of the present invention can encode a prokaryotic polymerase, such as, but not limited to, an SP6, T3, or T7 polymerase. In these aspects, one or more filamentous phage genes that are stably comprised by a bacterial strain of the present invention can be under the control of the prokaryotic promoter encoded on a phage display expression vector of the present invention, such that expression of the genes is precluded until the strain is transformed with the phage display expression vector.

The present invention contemplates, among other applications, the direction of the methods of the present invention toward the expression of antibodies and fragments thereof. In this regard, the display of immunoglobulin molecules that comprise at least a portion of both a heavy chain and at least a portion of a light chain is of particular interest. Strategies for displaying assembled heavy and light chains (or portions thereof) on the surface of a filamentous phage are known in the art, and all include the use of helper phage. They include, as non-limiting examples, the use of a phagemid vector that comprises sequences encoding the variable regions of heavy and light chains connected by a flexible linker (to create a “single chain variable region antibody” or “scFv”) and fused to cpIII sequences Dubel et al. Gene 128: 97-101 (1993), incorporated by reference) Alternate strategies for the expression of antibody molecules on the surface of filamentous phage make use of a plasmid vector that can contain a gene for an immunoglobulin heavy chain fused to a secretory leader peptide (a “soluble heavy chain”) and a phagemid vector that can contain a gene that encodes a fusion protein of an immunoglobulin light chain with a coat protein (or vice-versa, that is, soluble light chain, and heavy chain-coat protein fusion). These methods involve the transformation of bacteria with both the plasmid and the phagemid constructs, such that antibody molecules can assemble in the periplasm and be expressed on the surface of filamentous phage (see, for example, PCT/GB91/01134 WO92/01047, incorporated by reference).

In yet other antibody display technologies, a single phage display expression vector can express both the soluble heavy (or light) chain and light (or heavy) chain-coat protein fusions (PCT/GB91/01134 WO92/01047, incorporated by reference). And in still other antibody display technologies, the soluble heavy (or light) chain and light (or heavy) chain-coat protein fusion can be introduced into bacteria on different vectors (so that they can originate from different libraries), and can be induced to recombine into a single vector, for example, through use of the cre-lox recombination system (see, for example, U.S. Pat. No. 6,225,447 issued May 1, 2001 to Winter et al., incorporated by reference in its entirety).

Phage display constructs in which the heavy and light chains can be encoded on the same vector are particularly favored by the inventors, as it ensures their packaging within the same phage particle, and thus promotes the efficient isolation of optimal combinations of heavy and light chains. Systems that do not include supplying a wild-type coat protein that corresponds to the fusion coat protein are also preferred by the inventors, so that the display of the fusion protein on the phage particle is more likely. These features are advantageously combined with the use of a bacterial strain of the present invention that obviates the need for a helper phage to increase the efficiency of isolating antibodies with desirable characteristics.

A bacterial strain of the present invention can be transformed with a phage display expression vector by any convenient means (for example, calcium chloride treatment/heat shock or electroporation), and the production of phage particles can be induced in any of a variety of ways. In some embodiments of the present invention, transformation of the bacterial strain and expression of genes on the phage display expression construct will initiate phage production. In an alternative preferred embodiment of the present invention, the packaging genes that are integrated into the bacterial host strain chromosome and are regulated by a lac, ptac, or T7lac promoter, and phage production is induced by the addition of IPTG Sambrook et al., Molecular Cloning: A Laboratory Manual, 2nd edition, Cold Spring Harbor Press, Cold Spring Harbor, N.Y. (1989). It is also possible to have packaging genes regulated by rha or ara promoters, and induced by the addition of rhamnose or arabinose (Haldiman et al. J. Bact. 180: 1277-1286; Holcroft and Egan, J. Bact. 182: 6774-6782 (2000)). In particularly preferred aspects, different packaging function genes are regulated by different promoters (such as, but not limited to, a combination of those listed), and can be induced with different compounds or conditions. In this way, different packaging genes can be expressed at different levels to provide optimal levels of phage packaging functions.

Methods of culturing bacteria for filamentous phage production are well-known in the art (see, for example, Kay, Winter, and McCafferty, eds., Phage Display of Peptides and Proteins, A Laboratory Manual, Academic Press: San Diego (1996); Sambrook et al., Molecular Cloning: A Laboratory Manual, 2nd edition, Cold Spring Harbor Press, Cold Spring Harbor, N.Y. (1989); and C. F. Barbas III, D. R. Burton, J. K. Scott, and G. J. Silverman, Phage Display. A Laboratory Manual, Cold Spring Harbor, N.Y. (2001)). The phage-containing supernatant can be harvested from the bacterial culture and the phage can optionally be concentrated, for example, with PEG/NaCl, and optionally further purified on a CsCl gradient (see Rider et al. pp. 55-65 in Phage Display of Peptides and Proteins ed. by Kay et al. Academic Press, San Diego (1996), herein incorporated by reference).

Phage that bind or interact with a substance of interest can be selected for using such methods as panning procedures, ELISA, chromatography, fluorescence activated sorting, or enzymatic assays, all of which are well known in the art (see, for example, U.S. Pat. No. 5,679,548 issued to Barbas et al., Oct. 21, 1997, Janda et al., Proc. Natl. Acad. Sci. USA 91:2532-2536 (1994); Hart et al., J. Biol Chem. 269: 12468-74 (1994); Zaccolo et al., Eur. J. Immunol. 27: 618-623 (1997); Xie et al., Nat. Biotechnol. 15: 721-2 (1997); all herein incorporated by reference. Panning procedures can use ligands or substances of interest bound to a solid support, or can rely on immunoprecipitation, or sedimentation or collection after binding to cell surfaces or even viral surfaces, including the surfaces of other bacteriophage. Phage particles expressing display peptides or proteins that bind or interact with a substance of interest can also be separated by any other means that distinguishes binding or interacting display peptides and proteins from those that do not bind or interact.

Preferably, selected phage are amplified by using them to infect a bacterial host strain of the present invention, but this is not a requirement of the present invention. Optionally but preferably, the host strain bacteria containing the amplified phage are re-selected and then purified by plating on a bacterial host strain of the present invention and isolation of phage from single plaques using techniques known in the art (Sambrook et al., Molecular Cloning: A Laboratory Manual, 2nd edition, Cold Spring Harbor Press, Cold Spring Harbor, N.Y. (1989)). Preferably, a phage display expression vector of the present invention includes a selectable marker gene and plating can be done under selective conditions. Phage isolated from single plaques are cultured on host bacteria and can be used for the isolation of at least one phage display expression construct DNA molecule (Kay, Winter, and McCafferty, eds., Phage Display of Peptides and Proteins, A Laboratory Manual, Academic Press: San Diego (1996); C. F. Barbas III, D. R. Burton, J. K. Scott, and G. J. Silverman, Phage Display. A Laboratory Manual, Cold Spring Harbor, N.Y. (2001)). Phage display expression construct DNA can be sequenced to identify the amino acid sequence of the display peptide or protein.

One or more steps of the method can be performed reiteratively. For example, in some preferred embodiments filamentous bacteriophage that bind or interact with a sequence of interest can be isolated and used to reinfect a new batch of a bacterial strain of the present invention, and the bacterial strain can be used to produce more bacteriophage that can be used for the isolation of nucleic acid, or can be subjected to a second selection procedure before isolating the nucleic acid molecules contained by the phage, before or after another amplification in the bacterial strain host. This process is called enrichment of binding partners.

III. Peptides and Proteins Identified Using Bacterial Strains Supplying Packaging Functions in Phage Display

The present invention also includes peptides or proteins identified by the methods of the present invention. The peptides or proteins identified using the methods of the present invention can be any peptides or proteins, known or unknown, and can be encoded by nucleic acids derived from populations of nucleic acids from one or more organisms, or chemically synthesized. They may be known peptides or proteins that comprise novel or altered regions.

The peptides or proteins identified by the methods of the present invention can be, as nonlimiting examples, immunoglobulin molecules, receptors, ligands, enzymes, allosteric effector proteins, structural proteins, hormones, extracellular matrix proteins, signaling molecules, or any domain or portion thereof, or any combination thereof.

The peptides or proteins identified using the methods of the present invention can be used as research reagents or for industrial or commercial purposes, or can be part of a therapeutic or diagnostic formulation.

Pharmaceutical Compositions

The present invention also encompasses a peptide or protein identified using the methods of the present invention, or a portion or derivative thereof, in a pharmaceutical composition comprising a pharmaceutically acceptable carrier prepared for storage and preferably subsequent administration, which have a pharmaceutically effective amount of the peptide or protein in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The peptide or protein of the present invention can be formulated and used in tablets, capsules or elixirs for oral administration; suppositories for rectal administration; sterile solutions, suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparation, such as liposomes, can be used.

The pharmaceutically effective amount of a peptide or protein required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperitoneally, employing a variety of dosage forms. Such methods can also be used in testing the activity of peptide or protein of the present invention in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above, and can be guided by agencies such as the USFDA or NIH. Typically, human clinical applications of products are commenced at lower dosage levels, with dosage level being increased until the desired effect is achieved. Alternatively, acceptable in vitro studies can be used to establish useful doses and routes of administration of the peptides or proteins.

In non-human animal studies, applications of the pharmaceutical compositions are commenced at higher dose levels, with the dosage being decreased until the desired effect is no longer achieved or adverse side effects are reduced of disappear. The dosage for the peptides or proteins of the present invention can range broadly depending upon the desired affects, the therapeutic indication, route of administration and purity and activity of the test compound. Typically, dosages can be between about 1 ng/kg and about 10 mg/kg, preferably between about 10 ng/kg and about 1 mg/kg, more preferably between about 100 ng/kg and about 100 micrograms/kg, and most preferably between about 1 microgram/kg and about 10 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ disfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient, including those for veterinary applications.

Depending on the specific conditions being treated, such pharmaceutical compositions can be formulated and administered systemically or locally. Techniques for formation and administration can be found in Remington's Pharmaceutical Sciences, 18th Ed., Mack Publishing Co., Easton, Pa. (1990). Suitable routes of administration can include oral, nasal, rectal, transdermal, otic, ocular, vaginal, transmucosal or intestinal administration; parenteral delivery, including intramuscular, subcutaneous, intramedullary injections, as well as intrathecal, direct intraventricular, intravenous, intraperitoneal, intranasal, or intraocular injections.

For injection, the pharmaceutical compositions of the present invention can be formulated in aqueous solutions, preferably in physiologically compatible buffers such as Hanks' solution, Ringer's solution or physiological saline buffer. For such transmucosal administration, penetrants appropriate to the barrier to be permeated are used in the formulation. Such penetrants are generally known in the art. Use of pharmaceutically acceptable carriers to formulate the pharmaceutical compositions herein disclosed for the practice of the invention into dosages suitable for systemic administration is within the scope of the invention. With proper choice of carrier and suitable manufacturing practice, the compositions of the present invention, in particular, those formulation as solutions, can be administered parenterally, such as by intravenous injection. The pharmaceutical compositions can be formulated readily using pharmaceutically acceptable carriers well known in the art into dosages suitable for oral administrations. Such carriers enable the test compounds of the invention to be formulated as tables, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Intracellular delivery of drugs may be acheived by linking peptides such as the translocating domain of the tat protein of HIV to the agent. Linkage of hydrophobic molecules such as biotin to the attached tat peptide or similar translocating peptides may improve intracellular delivery further (Chen et al. Analyt. Biochem. 227: 168-175 (1995)). Substantially all molecules present in an aqueous solution at the time of liposome formation are incorporated into or within the liposomes thus formed. The liposomal contents are both protected from the external micro-environment and, because liposomes fuse will cell membranes, are efficiently delivered into the cell cytoplasm. Additionally, due to their hydrophobicity, small organic molecules can be directly administered intracellularly.

Pharmaceutical compositions suitable for use in the present invention include compositions wherein the active ingredients are contained in an effective amount to achieve its intended purpose. Determination of the effective amount of a pharmaceutical composition is well within the capability of those skilled in the art, especially in light of the detailed disclosure provided herein. In addition to the active ingredients, these pharmaceutical compositions can contain suitable pharmaceutically acceptable carriers comprising excipients and auxiliaries which facilitate processing of the active chemicals into preparations which can be used pharmaceutically. The preparations formulated for oral administration may be in the form of tables, dragees, capsules or solutions. The pharmaceutical compositions of the present invention can be manufactured in a manner that is itself known, for example by means of conventional mixing, dissolving, granulating, dragee-making, emulsifying, encapsulating, entrapping or lyophilizing processes. Pharmaceutical formulations for parenteral administration include aqueous solutions of active chemicals in water-soluble form.

Additionally, suspensions of the active chemicals may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances what increase the viscosity of the suspension, such as sodium carboxymethyl cellulose, sorbitol or dextran. Optionally, the suspension can also contain suitable stabilizers or agents that increase the solubility of the chemicals to allow for the preparation of highly concentrated solutions.

Pharmaceutical compositions for oral use can be obtained by combining the active chemicals with solid excipient, optionally grinding a resulting mixture, and processing the mixture of granules, after adding suitable auxiliaries, if desired, to obtain tables or dragee cores. Suitable excipients are, in particular, fillers such as sugars, including lactose, sucrose, mannitol or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose and/or polyvinylpyrrolidone. If desired, disintegrating agents can be added, such as the cross-linked polyvinyl pyrolidone, agar, alginic acid or a salt thereof such as sodium alginate. Dragee cores can be provided with suitable coatings. Dyes or pigments can be added to the tablets or dragee coatings for identification or to characterize different combinations of active doses.

The peptides or proteins of the present invention, and pharmaceutical compositions that include such peptides and proteins, or portions thereof, can be used to treat a variety of ailments in a patient, including a human. The peptides or proteins of the present invention can have antibacterial, antimicrobial, antiviral, anticancer cell, antitumor and cytotoxic activity. A patient in need of such treatment can be provided a peptide or protein of the present invention, or a portion thereof, preferably in a pharmacological composition. The amount, dosage, route of administration, regime and endpoint can all be determined using the procedures described herein or by appropriate government agencies, such as the United Stated Food and Drug Administration.

### EXAMPLES

### Example 1

### Construction of Transfer Plasmid Constructs Containing M13 Gene Modules

Four M13 gene modules are constructed separately into transfer plasmids that contain the following elements: 1) the conditional origin of replication ori R6Kγ(gamma) that is only able to function in cells producing π (pi) protein, 2) a regulatable promoter (the araB, rhaB, rhaS, or lac promoter; Haldiman et al. J. Bact. 180: 1277-1286 (1998), Holcroft and Egan J. Bact. 182: 6774-6782 (2000)), 3) a multiple cloning site containing unique six-nucleotide cleavage sites, 4) an attachment site (att) for one of the five lambdoid phages, 5) an antibiotic resistance cassette (encoding genes conferring resistance to streptomycin, chloramphenicol, gentamycin, kanamycin, or tetracycline, and 5) appropriately positioned transcription terminators (t_{L3} and t₁t₂) that act as insulators to enable the different transcription units to function automomously. In making each transfer plasmid construct, these elements are cloned in the orientation shown in FIG. 1b.

M13 genes II/X and V that encode M13 DNA replication functions are cloned into the transfer plasmid construct 1 (see FIG. 1c for Module 1, consisting of the P(araB) promoter, genes II/X and V, and the tL3 terminator). A streptomycin resistance gene is cloned into plasmid construct 1 at the ‘antR’ position depicted in FIG. 1b.

M13 genes VI, VII, and IX that encode M13 minor capsid proteins are cloned into the transfer plasmid construct 2 (see FIG. 1c for Module 2, consisting of the P(rhaB) promoter, genes VI, VII, and IX, and the tL3 terminator). A chloramphenicol resistance gene is cloned into plasmid construct 2 at the ‘antR’ position depicted in FIG. 1b.

M13 genes I/XI, and IV that encode M13 assembly and secretion proteins arecloned into the transfer plasmid construct 3 (see FIG. 1c for Module 3, consisting of the P(lac) promoter, genes I/XI, and IV, and the tL3 terminator). A gentamycin resistance gene is cloned into plasmid construct 3 at the ‘antR’ position depicted in FIG. 1b.

Four copies of M13 gene VIII that encodes the M13 major capsid protein will be cloned into the transfer plasmid construct 4 (see FIG. 1b for Module 4, consisting of the P(rhaS) promoter, four copies of gene VIII, and the tL3 terminator). A tetracycline resistance gene is cloned into plasmid construct 4 at the ‘antR’ position depicted in FIG. 1b.

Cloning operations will be performed by amplifying M13 genes with primers that contain unique restriction sites such that amplified products can be ligated into the constucts in the appropriate order. Transfer plasmid intermediates and final constructs will be transformed into a bacterial host that expresses the π (pi) protein.

### Example 2

### Integration of M13 Modules into the Bacterial Chromosome

The transfer plasmid constructs will be sequentially introduced into an E. coli host that does not express the π (pi) protein by transformation. The E. coli host strain harbors the pLDR8 plasmid (construction and use of this plasmid is described in Diederich et al. Plasmid 28: 14-24 (1992), incorporated by reference) that contains the lambda int gene and C_{I 857} gene encoding the lambda repressor under the control of the lambda P_{R} promoter. The pLDR8 plasmid has a temperature sensitive origin of replication that prevents replication at 42 degrees centigrade, and contains a kanamycin resistance gene.

The E. coli host will be grown at 30 degrees C. in the presence of kanamycin to allow replication of the pLDR8 plasmid. The culture is then diluted 1:20 in media containing 50 micrograms per ml kanamycin and glucose at 37 degrees C. After 90 min the cells are transformed with transfer plasmid 1 using CaCl₂ solution and a 42 degree C. heat shock. Following transformation with transfer plasmid construct 1, the cells are plated on media containing glucose (to repress expression of phage genes from ara, rha, or lac promoters in the absence of kanamycin, and in the presence of streptomycin to select for the transfer plasmid, and incubated at 42 degrees. The elevated temperature derepresses the lambda P_{R} promoter by inactivating the CI ₈₅₇ repressor. Thus, the lambda integrase is expressed to promote integration of the transfer construct. The elevated temperature also prevents replication of the pLDR8 plasmid, so that the pLDR8 plasmid is lost and expression of the lambda integrase is curtailed.

Transformed bacteria that grow in the presence of antibiotic should have the transfer plasmid in integrated form, since the absence of the π (pi) protein in the host prevents replication of the transfer plasmid. Integration of the transfer construct at an att locus can be confirmed by PCR with locus-specific primers and diagnostic restriction enzyme digests of the PCR products. Each transfer construct is introduced into the bacterial strain in the same way, with the appropriate antibiotic added in each case to select for the particular transfer construct.

### Example 3

### Production of M13 Particles by a Genetically Engineered Bacterial Strain

The bacterial strain that has the four integrated constructs comprising M13 genes is grown in the presence of glucose to repress the transcription of the M13 packaging function genes from the lac, ara, and rha promoters. The bacterial strain is transformed with a phagemid vector that contains an immunoglobulin light chain-cpIII fusion protein gene under the control of the lac promoter, and immunoglobulin heavy chain gene also under the control of the lac promoter, and the bla gene for ampicillin resistance.

Expression of M13 proteins and the light chain-cpIII fusion protein and heavy chain protein and the synthesis of phage particles is induced by the removal of glucose from the growth media and the addition of IPTG, rhamnose, and arabinose. Phage particles can be harvested from the media of the bacterial culture and used in phage display selection protocols.

### Example 4

### Construction of Transfer Plasmid Constructs Containing Filamentous Phage M13 Gene Modules

Cloning of M13 Packaging Genes

M13 packaging genes were cloned in four separate groups: Group 1: genes I, IV, and XI (2.3 kb); Group 2: genes II, V, and X (1.5 kb); Group 3: gene VI (340bp); Group 4: genes VII and IX (200 bp); and Group 5: gene VIII (220 bp). Each group of genes was amplified using the M13 genome as a template the primers listed in Table 1:

TABLE 1
Primer sets used for PCR amplification of gene fragments

| | |
|---|---|
| Group 1 | Forward 5′-catatg gct gtt tat ttt-3′ |
| | (SEQ ID NO:1) |
| Genes I, IV, and XI | Reverse 5′-gct agc cta cag ggc gcg tac-3′ |
| | (SEQ ID NO:2) |
| Group 2 | Forward 5′-cat atg att gac atg cta-3′ |
| | (SEQ ID NO:3) |
| Genes II, V, and X | Reverse 5′-gct agc tta ctt agc cgg gaa c-3′ |
| | (SEQ ID NO:4) |
| Group 3 | Forward 5′-cat atg cca gtt ctt ttg-3′ |
| | (SEQ ID NO:5) |
| Gene VI | Reverse 5′-gct agc tta ttt atc cca atc-3′ |
| | (SEQ ID NO:6) |
| Group 4 | Forward 5′-cat atg gag cag gtc gcg-3′ |
| | (SEQ ID NO:7) |
| Genes VII and IX | Reverse 5′-gct agc tca tga gga agt ttc-3′ |
| | (SEQ ID NO:8) |
| Group 5 | Forward 5′-cat atg aaa aag ctt ta-3′ |
| | (SEQ ID NO:9) |
| Gene VIII | Reverse 5′-gct agc tca gct tgc ttt cga-3′ |
| | (SEQ ID NO:10) |

The following conditions were used in the amplification reactions: 2 units of Taq or Pfu polymerase was added to 50 ng of M13 mp18 template and 15 picomoles each of the appropriate forward and reverse primers. The reactions contained 1.5 millimolar MgCl₂, 10 millimolar Tris-HCl, pH 8.5, 100 millimolar KCl, and 100 micromolar each of dATP, dCTP, dGTP, and dTTP in a total volume of 40 microliters. The reactions were heated to 95 degrees C. for 5 min, cooled to 52 degrees C. for 30 seconds, and extended at 72 degrees C. for 1 min., after which 35 cycles were performed as follows: 94 degrees C. for 30 seconds, 52 degrees C. for 30 seconds, and 72 degrees C. for 1 min. After the final cycle, the reactions were left at 4 degrees C. overnight.

The amplification products were examined by gel electrophoresis and fragments of the expected size were observed in all cases. Each of the fragments originating from amplification of a segment of the M13 genome was cloned into a TA cloning vector (Invitrogen, Calsbad, Calif.) and their sequences were verified by DNA sequencing.

Integrations of Gene Modules into the E. coli Chromosome.

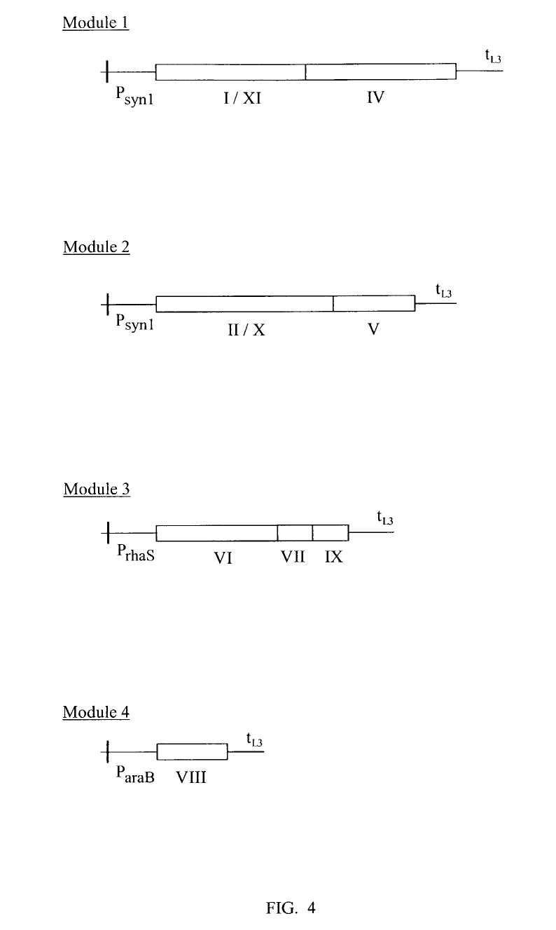

The M13 gene groups were excised from the TA cloning vectors using the restriction enzymes listed in Table 2. They were then ligated into CRIM plasmids digested with the same enzymes to form modules for integration into the host chromosome. Module 1 contained the genes of Group 1 and Module 2 contained the genes of Group 2. Module 3 was constructed to contain the genes of both Group 3 and Group 4. In the case of Module 4, the ara promoter was ligated into CRIM plasmid pAH162 along with M13 gene VIII (Group 5) as part of the same PstI-XmaI fragment (see FIG. 4).

CRIM plasmids will replicate only in hosts expressing the II protein encoded by the pir gene. A series of CRIM plasmids were used, each having an antibiotic resistance gene, a promoter, and a polylinker for cloning, a phage attachment (attP) sites, bacterial and phage transcription terminators, and a conditional origin of replication. CRIM plasmids and methods of using CRIM plasmids (including E. coli chromosomal integration hosts and methods) are described in Haldiman and Wanner, J. Bacteriol. 183: 6384-6393 (2001), incorporated by reference in its entirety. The CRIM plasmids were transformed into and propagated in the host strains given in Table 2 using standard molecular biology methods.

TABLE 2
M13 gene modules cloned in CRIM plasmids.

| | | | | | | Antibiotic and |
|---|---|---|---|---|---|---|
| | | | Inserted | Endo- | | restriction |
| CRIM | | | DNA | nuclease | Recipient | mapping |
| plasmid | Promotor | Inducer | fragment | sites | bacterium | check results |
| PAH95 | Psyn1 | None | M13 Gene | NdeI, | BW23474 | kan resistant |
| Module 1 | | | I, IV, XI | BamHI | | resmap corr. |
| PCAH63 | Psyn1 | None | M13 Gene | XbaI, | BW25141 | kan. resistant |
| Module 2 | | | II, V, X | BamHI | | resmap corr. |
| pAH152 | PrhaS | 6-Deoxy-D- | M13 Gene | NdeI, | BW23474 | gen resistant |
| Module 3 | | glucose | VI, | BamHI/ | | resmap corr. |
| | | | VII, IX | BamHI, | | |
| | | | | EcoRI | | |
| pAH162 | ParaB | L-(+)- | ParaB + | PstI&XmaI | BW25141 | tet resistant |
| Module 4 | | Arabinose | Gene | | | resmap corr. |
| | | | VIII | | | |

Host strain BW25141 and CRIM vectors are described in Haldiman and Wanner J. Bacteriol. 183: 6384-6393 (2001).
Host strain BW23474 is described in Haldiman et al. Proc. Natl. Acad. Sci. USA 93: 14361-14366 (1996).
gen is gentamycin, kan is kanamycin, and tet is tetracycline.
“resmap corr.” means the predicted restriction map was confirmed by digests.

Integration of M13 Gene Modules into Host Chromosome

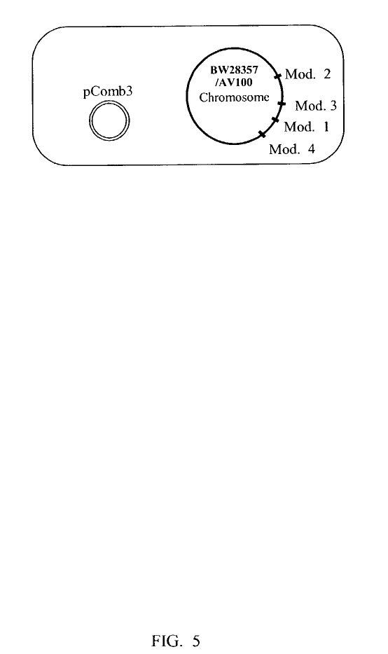

For integration of M13 gene modules into the E.coli host, CRIM plasmids were transformed stepwise into the host BW28357, a pyrE+ derivative of BW25113 (Haldiman and Wanner J. Bacteriol. 183: 6384-6393 (2001)), constructed by P1 transduction. (This alteration was made in light of the realization that many E. coli strains are leaky pyrE mutants, as described in Jensen, J. Bacteriol. 175: 3401-3407 (1993)). The BW28357 strain had a CRIM helper plasmid that, at elevated temperature, expresses the int gene for integration of plasmid inserts at attB sites and does not express the II protein necessary for CRIM plasmid replication. In each step, a single CRIM plasmid carrying an M13 gene module was introduced into the host and colonies were selected for the appropriate antibiotic resistance. The transformation procedure involved growing the host cells that contained the helper plasmid in SOC media and ampicillin (to select for helper plasmid) at 30 degrees C. to an OD 600 of approximately 0.6. The cells were electroporated in the presence of a CRIM plasmid according to standard procedures, after which cells were grown in nonselective SOC at 37 degrees C. for 1 h and then at 42 degrees C. for 30 min. The elevated temperature induces expression of the int gene from the helper plasmid while at the same time inhibiting replication of the helper plasmid so that it is lost. The cells were plated on media containing the antibiotic for which the CRIM plasmid used in transformation had a resistance gene. Resistant colonies were grown up and their DNA examined for the presence of the introduced M13 gene module at the targeted att locus. A clone having an appropriate integrated M13 module was then used as a transformation host for the next CRIM plasmid carrying another M13 gene module. Selection was based on double antibiotic resistance (resistance from the first introduced plasmid plus the newly introduced one). By this method, all five modules in different CRIM plasmids were introduced into the chromosome of same host: Module 1 was integrated into the P21 att site, Module 2 was integrated into the lambda att site, Module 3 was integrated into the HK att site, and Module 4 was integrated into the 80 att site (see FIG. 5). Final constructions were verified for correctness by PCR and antibiotic resistances. All genes in the four modules were successfully integrated onto the host BW 28357 to give the engineered strain BW28357/AV100.

Phagemid Packaging by Engineered Strain BW28357/AV100

To test the phage packaging function of BW28357/AV100, the following experiments were carried out.

To test the phage packaging function of BW28357/AV100, a phagemid containing an M13 gene III chimeric protein was transformed into the strain. The strain was then tested for the ability to produce new phage particles containing the phagemid DNA.

BW28357/AV100 cells were transformed by the calcium chloride method with phagemid pComb3 (Barbas et al., Proc. Natl. Acad. Sci. USA 88: 7978-7982 (1991)). Phagemid pComb3 contains the IgG heavy chain gene joined by a linker to a portion of M13 gene III.

The cells of a single colony were grown at 37 degrees C. in 10 ml of LB medium containing 50 microgram/ml of each antibiotic plus ampicillin to approximately OD600 of 0.6. 10 microliters of a solution of 6-Deoxy-D-glucose and 10 microliters of a solution of L-(+)-Arabinose were added to the culture, which was allowed to incubate overnight.

The supernatant was collected by centrifugation to remove bacteria. 0.2 volumes of 30% PEG in 2 M NaCl were added to the supernatant to precipitate the assembled phage particles. After incubating the supernatant for 15 min on ice, the PEG/supernatant was centrifuged at 8,000 ×g for 10 min. The supernatant was discarded, and the pellet was resuspended in 50 mM Tris, pH 8, 10 mM EDTA and 1% SDS. The resuspension was heated at 90 degrees C. for 5 minutes, and the supernatant was phenol/CHCl3 extracted twice and CHCl3 extracted once. The phagemid DNA of the final aqueous phase was ethanol precipitated and the pellet resuspended in 10 mM Tris, pH 8, 1 mM EDTA. The OD 260 reading was 0.3. The DNA was subject to PCR and restriction enzyme digestion.

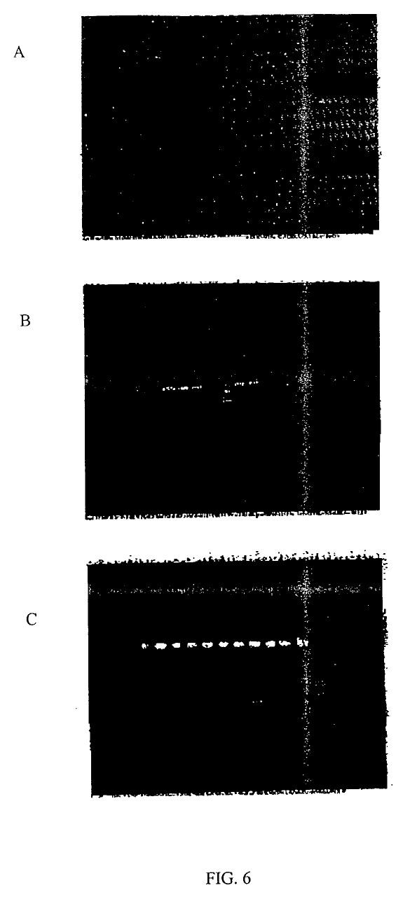

PCR to test for the presence of phagemid DNA was performed according to standard protocols. The primers used were pComb3 specific primers: 5′-gaggaggaggaggaggaggtggcccaggcggccctgactcag-3′ (CSC-F sense) (SEQ ID NO: 11) and 5′-gaggaggaggaggaggaggagagaagcgtagtccggaacgtc-3′ (dp-EX antisense) (SEQ ID NO: 12). The gel showed PCR fragments corresponding to phagemid DNA as shown in FIG. 6.

Restriction enzyme digestion was performed on the phagemid DNA prep to confirm the presence of phagemid DNA using Xho I and Xba I. Gel electrophoresis showed the expected restriction fragments.

Thus, host BW28357/AV100 has all of the packaging functions of an M13 phage.

All publications, including patent documents and scientific articles, referred to in this application, including any bibliography, are incorporated by reference in their entirety for all purposes to the same extent as if each individual publication were individually incorporated by reference. All headings are for the convenience of the reader and should not be used to limit the meaning of the text that follows the heading, unless so specified.

Bibliotraphy

U.S. Pat. No. 5,258,498 issued to Huston et al., Nov. 2, 1993.

U.S. Pat. No. 5,270,163 to Gold et al., issued Dec. 14, 1993.

U.S. Pat. No. 5,667,988 issued to Barbas et al., Sep. 16, 1997.

U.S. Pat. No. 5,679,548 issued to Barbas et al., Oct. 21, 1997.

U.S. Pat. No. 5,741,657 to Tsien et al., issued Apr. 21, 1998.

U.S. Pat. No. 5,747,253 to Ecker et al., issued May 5, 1998.

U.S. Pat. No. 5,777,079 to Tsien et al., issued Jul. 7, 1998.

U.S. Pat. No. 5,804,387 to Cormack et al., issued Sep. 8, 1998.

U.S. Pat. No. 5,908,626 issued to Chang et al., Jun. 1, 1999.

U.S. Pat. No. 6,225,447 issued to Winter et al., May 1, 2001.

PCT/GB91/01134 WO92/01047

PCT/US99/22436 (WO 00/18778), Lohse et al., published Apr. 6, 2000

Ausubel et al., Current Protocols in Molecular Biology, John Wiley and Sons (1998).

Barbas et al., Phage Display. A Laboratory Manual, Cold Spring Harbor, N.Y. (2001).

Barbas et al. Proc. Natl. Acad. Sci. 88: 797807982 (1991).

Chen et al. Analyt. Biochem. 227: 168-175 (1995).

Diederich et al. Plasmid 28: 14-24 (1992).

Dubel et al. Gene 128: 97-101 (1993).

Dubendorff and Studier J. Mol. Biol. 219: 45-59 (1991).

Fingle et al., in The Pharmacological Basis of Therapeutics (1975).

Frischer et al., Curr Microbiol 33: 287-291 (1996).

Frischer et al., Appl. Environ Microbiol 56: 3439-3944 (1990).

Haldiman et al., Proc. Natl. Acad. Sci. USA 93:14361-14366 (1996).

Haldiman et al. J. Bacteriol. 180: 1277-1286 (1998).

Haldiman and Wanner J. Bacteriol. 183: 6384-6393 (2001).

Harlow and Lane, Antibodies, A Laboratory Manual, Cold Spring Harbor Press (1988).

Hart et al., J. Biol Chem. 269: 12468-74 (1994).

Holcroft and Egan, J. Bact. 182: 6774-6782 (2000).

Janda et al., Proc. Natl. Acad. Sci. USA 91:2532-2536 (1994).

Jensen, K. F., J. Bacteriol. 175: 3401-3407 (1993).

Kay, Winter, and McCafferty, eds., Phage Display of Peptides and Proteins. A Laboratory Manual, Academic Press: San Diego (1996).

Mathys et al. Gene 231: 1-13 (1999).

Mercer et al. FEMS Microbiol. Lett. 179: 485-490 (1999).

Nakamura and Inouye EMBO J. 1: 771-775 (1982).

Niedhart, et al., Escherichia coli and Salmonella: Cellular and molecular biology ASM Press, Washington D.C. (1996).

Pasqualini and Ruoslahti, Nature 380:364-366 (1999).

Remington's Pharmaceutical Sciences, 18th Ed., Mack Publishing Co., Easton, Pa. (1990).

Sambrook et al., Molecular Cloning: A Laboratory Manual, 2nd edition, Cold Spring Harbor Press, Cold Spring Harbor, N.Y. (1989).

Saunders et al. Microbiol. 145: 3523-3528 (1999).

Sikorski et al., Appl. Environ Microbiol. 68: 865-873 (2002).

Stewart and Sinigalliano, AntonieVan Leeuwenhoek 59: 19-25 (1991).

Viera and Messing, Production of Single-Standed Plasmid DNA in Methods in Enzymology vol. 153, ed. Wu and Grossman, Academic Pres, New York, pp 3-11 (1987).

Xie et al., Nat. Biotechnol. 15: 721-2 (1997).

Zaccolo et al., Eur. J. Immunol. 27: 618-623 (1997).

We claim:

1. A bacterial strain stably comprising one or more genes that encodes a packaging function of a filamentous bacteriophage, wherein said one or more genes can, together with at least one other gene that resides on a phage display expression vector, produce and package said phage display expression vector in an infectious filamentous phage particle in the absence of a helper phage.

2. The bacterial strain of claim 1, wherein said bacterial strain has an F factor.

3. The bacterial strain of claim 2, wherein said bacterial strain is an E. coli strain.

4. The bacterial strain of claim 1, wherein said at least one gene is integrated into bacterial host chromosome.

5. The bacterial strain of claim 4, wherein at least one of said one or more genes is integrated at an attB site.

6. The bacterial strain of claim 5, wherein said one or more genes comprises at least one of M13 genes I-XI, fd genes I-XI, or fl genes I-XI.

7. The bacterial strain of claim 6, wherein said one or more genes is ten genes.

8. The bacterial strain of claim 7, wherein said ten genes are organized into at least one module, wherein said at least one module comprises at least one copy of one or more of said ten genes.

9. The bacterial strain of claim 8, wherein said ten genes are organized into two or more modules.

10. The bacterial strain of claim 9, wherein each of said two or more modules comprises an inducible promoter that regulates, at least in part, all of the genes of the module.

11. The bacterial strain of claim 10, wherein said inducible promoter is inducible by a small molecule.

12. The bacterial strain of claim 11, wherein said small molecule is a sugar, a sugar analogue, a nucleotide, a nucleotide analogue, an antibiotic, or a pheremone.

13. The bacterial strain of claim 10, wherein each of at least two of said two or more modules comprises a different inducible promoter.

14. The bacterial strain of claim 13, wherein said different inducible promoters have different strengths.

15. The bacterial strain of claim 14, wherein said ten genes are M13 genes I-VII, IX, X, and XI.

16. The bacterial strain of claim 14, wherein said ten genes are M13 genes I, II, and IV-XI.

17. The bacterial strain of claim 16, wherein said ten genes are organized into four modules that are integrated into four sites of said chromosome.

18. The bacterial strain of claim 17, wherein one module comprises M13 genes I, IV, and XI, one module comprises M13 genes II, V, and X, one module comprises M13 gene VI, VII and IX, and one module comprises M13 gene VIII.

19. The bacterial strain of claim 18, designated BW28357/AV100.

20. A bacterial strain stably comprising one or more genes that encodes a packaging function of a filamentous bacteriophage, wherein said one or more genes can produce and package a phage display expression vector in an infectious filamentous phage particle in the absence of a helper phage.

21. The bacterial strain of claim 20, wherein said bacterial strain has F factor.

22. The bacterial strain of claim 21, wherein said bacterial strain is an E. coli strain.

23. The bacterial strain of claim 22, wherein said one or more genes is integrated into bacterial host chromosome.

24. The bacterial strain of claim 23, wherein said one or more genes comprises M13 genes I-XI, fd genes I-XI, or fl genes I-XI.

25. The bacterial strain of claim 24, wherein said one or more genes comprises M13 genes I-XI.

26. The bacterial strain of claim 25, wherein said one or more genes are organized into at least one module, wherein said at least one module comprises at least one copy of one or more of said one or more genes.

27. The bacterial strain of claim 26, wherein said one or more genes are organized into two or more modules.

28. The bacterial strain of claim 27, wherein each of at least two of said two or more modules comprises an inducible promoter that regulates, at least in part, all of the genes of the module.

29. The bacterial strain of claim 28, wherein said at least one inducible promoter is inducible by a small molecule.

30. The bacterial strain of claim 29, wherein said small molecule is a sugar, a sugar analogue, a nucleotide, a nucleotide analogue, a pheremone, or an antibiotic.

31. The bacterial strain of claim 29, wherein at least two of said two or more modules comprise different inducible promoters.

32. The bacterial strain of claim 43, wherein said different inducible promoters have different strengths.

33. A method of identifying a peptide or protein, comprising;
cloning a nucleic acid sequence encoding a random sequence or a sequence of interest into an expression vector that comprises an open reading frame that encodes at least a portion of a coat protein of a filamentous bacteriophage such that said random sequence or sequence of interest and said open reading frame that encodes at least a portion of a coat protein of a filamentous bacteriophage can form at least one fusion protein or chimeric protein;
transforming said expression vector into the bacterial strain of claim 1;
inducing said bacterial strain to produce bacteriophage;
isolating at least one bacteriophage that binds or interacts with at least one substance of interest;
isolating at least one nucleic acid molecule from said at least one bacteriophage that binds or interacts with at least one substance of interest;
sequencing said at least one nucleic acid molecule from said at least one bacteriophage that binds or interacts with at least one substance of interest; and
identifying at least one random peptide or protein or at least one peptide or protein of interest expressed on said bacteriophage.

34. The method of claim 33, wherein said expression vector comprises at least a portion of an M13, fd, or fl bacteriophage coat protein gene.

35. The method of claim 34, wherein said expression vector comprises at least a portion of M13 gene III, gene VIII, gene VII, gene IX, or gene VI.

36. The method of claim 35, wherein said expression vector comprises at least a portion of gene III or gene VIII.

37. The method of claim 33, wherein said inducing is by the addition of a small molecule.

38. The method of claim 37 wherein said small molecule is a sugar, or a sugar analogue, a nucleotide, a nucleotide analog, a pheremone, or an antibiotic.

39. The method of claim 33, wherein said identifying is by ELISA, panning, chromatography, FACS or detection of an enzymatic reaction.

40. A peptide or protein identified by the method of claim 33.


SEQUENCE LISTING (12 sequences)

SEQ ID NO: 1 - DNA, 18 bp, Bacteriophage M13
catatggctgtttatttt

SEQ ID NO: 2 - DNA, 21 bp, Bacteriophage M13
gctagcctacagggcgcgtac

SEQ ID NO: 3 - DNA, 18 bp, Bacteriophage M13
catatgattgacatgcta

SEQ ID NO: 4 - DNA, 22 bp, Bacteriophage M13
gctagcttacttagccgggaac

SEQ ID NO: 5 - DNA, 18 bp, Bacteriophage M13
catatgccagttcttttg

SEQ ID NO: 6 - DNA, 21 bp, Bacteriophage M13
gctagcttatttatcccaatc

SEQ ID NO: 7 - DNA, 18 bp, Bacteriophage M13
catatggagcaggtcgcg

SEQ ID NO: 8 - DNA, 21 bp, Bacteriophage M13
gctagctcatgaggaagtttc

SEQ ID NO: 9 - DNA, 17 bp, Bacteriophage M13
catatgaaaaagcttta

SEQ ID NO: 10 - DNA, 21 bp, Bacteriophage M13
gctagctcagcttgctttcga

SEQ ID NO: 11 - DNA, 42 bp, Artificial Sequence
  Synthetic Construct
gaggaggaggaggaggaggtggcccaggcggccctgactcag

SEQ ID NO: 12 - DNA, 39 bp, Artificial Sequence
  Synthetic Construct
gaggaggaggaggaggagagaagcgtagtccggaacgtc